SEQUENCE LISTING (25 sequences)

SEQ ID NO: 1 - DNA, 32 bp, Artificial Sequence
  MED15_CpG-Tag forward primer
ccactcactcacccacccgtagaaaatgtagg

SEQ ID NO: 2 - DNA, 32 bp, Artificial Sequence
  MED15_CpG-Tag reverse primer
gggtgggaggtgggagggaacacacaaataac

SEQ ID NO: 3 - DNA, 18 bp, Artificial Sequence
  Tag sequencing forward primer
ccactcactcacccaccc

SEQ ID NO: 4 - DNA, 18 bp, Artificial Sequence
  Tag sequencing reverse primer
gggtgggaggtgggaggg

SEQ ID NO: 5 - DNA, 21 bp, Artificial Sequence
  MED15_MSP5′ reverse primer
aaaaatcccacaatccaaccc

SEQ ID NO: 6 - DNA, 22 bp, Artificial Sequence
  MED15_MSP5′ forward unmethylated primer
gttttgtgattgaggyggtggt

SEQ ID NO: 7 - DNA, 22 bp, Artificial Sequence
  MED15_MSP5′ forward methylated primer
gttttgtgattgaggyggcggc

SEQ ID NO: 8 - DNA, 22 bp, Artificial Sequence
  MED15_MSP3′ forward primer
gatatgggtggtgggagttggg

SEQ ID NO: 9 - DNA, 22 bp, Artificial Sequence
  MED15_MSP3′ reverse methylated primer
aatcagaccctaacctcgcccg

SEQ ID NO: 10 - DNA, 27 bp, Artificial Sequence
  MyoD forward primer
tgattaatttagattgggtttagagaa

SEQ ID NO: 11 - DNA, 23 bp, Artificial Sequence
  MyoD reverse primer
ccaactccaaatcccctctctat

SEQ ID NO: 12 - DNA, 33 bp, Homo sapiens
gtgattgaggcggcggcggtggcggttaagcgg

SEQ ID NO: 13 - DNA, 33 bp, Homo sapiens
gtgattgaggtggtggtggtggtggttaagtgg

SEQ ID NO: 14 - DNA, 33 bp, Homo sapiens
gtgattgaggtggcggtggtggtggttaagtgg

SEQ ID NO: 15 - DNA, 33 bp, Homo sapiens; identical to SEQ ID NO: 13

SEQ ID NO: 16 - DNA, 33 bp, Homo sapiens
gtgattgaggtggcggcggtggtggttaagtgg

SEQ ID NO: 17 - DNA, 33 bp, Homo sapiens; identical to SEQ ID NO: 16

SEQ ID NO: 18 - DNA, 33 bp, Homo sapiens; identical to SEQ ID NO: 16

SEQ ID NO: 19 - DNA, 26 bp, Homo sapiens
ttagcgtggcgggcgaggttagggtc

SEQ ID NO: 20 - DNA, 26 bp, Homo sapiens
taaggggggtgggtggggtaggggtg

SEQ ID NO: 21 - DNA, 26 bp, Homo sapiens
tagggggggtgggtggggttggggtg

SEQ ID NO: 22 - DNA, 26 bp, Homo sapiens
tagggggggtgggcggggtaggtgtg

SEQ ID NO: 23 - DNA, 26 bp, Homo sapiens
taaggggggcgggcggggtaggggtg

SEQ ID NO: 24 - DNA, 26 bp, Homo sapiens
tagggggggcgggtggggttggggtg

SEQ ID NO: 25 - DNA, 26 bp, Homo sapiens
tagggggggcgggcggggtaggggtg

(19) United States
(12) Patent Application Publication
(10) Pub. No.: US 2019/0032143 A1
(43) Pub. Date: Jan. 31, 2019

(54) KITS AND METHODS FOR DIAGNOSIS, SCREENING, TREATMENT AND DISEASE MONITORING

(71) Applicant: Queensland University of Technology, Brisbane (AU)

(72) Inventors: Chamindie PUNYADEERA, Moggill (AU); Dmitry OVCHINNIKOV, Jamboree Heights (AU)

(21) Appl. No.: 15/950,889

(22) Filed: Apr. 11, 2018

Related U.S. Application Data
(63) Continuation of application No. 15/026,960, filed on Apr. 1, 2016, now abandoned.

(30) Foreign Application Priority Data
Oct. 1, 2013 (AU) 2013903793

(51) Int. Cl. C12Q 1/6886

(52) U.S. Cl. CPC C12Q 1/6886; C12Q 2600/154; C12Q 2600/118

(57) ABSTRACT

Disclosed are methods for detecting the presence of a carcinoma or an increased likelihood that a carcinoma is present in a subject. More particularly, the present invention discloses methods for diagnosis, screening, treatment and monitoring of carcinomas associated with aberrant DNA methylation of the MED15 promoter region.

This application is a continuation of application Ser. No. 15/026,960, 371(c) date Apr. 1, 2016, now abandoned; which is the U.S. national phase of International Application No. PCT/AU2014/050261, filed Oct. 1, 2014, which designated the U.S. and claims priority to Australian Provisional Application No. 2013903793 entitled “Kits and Methods for Diagnosis, Treatment and Disease Monitoring” filed Oct. 1, 2013, the contents of each of which are incorporated herein by reference in their entirety.

### REFERENCE TO SEQUENCE LISTING SUBMITTED ELECTRONICALLY

The content of the electronically submitted sequence listing in ASCII text file (Name: 0659.0199_Sequence_Listing.txt; Size: 4.6 KB; and Date of Creation: Oct. 9, 2018) filed on Oct. 9, 2018 is incorporated herein by reference in its entirety.

### FIELD OF THE INVENTION

This invention relates generally to methods for detecting the presence of a carcinoma or an increased likelihood that a carcinoma is present in a subject. More particularly, the present invention relates to methods for diagnosis, Screening, treatment and monitoring of carcinomas associated with aberrant DNA methylation of the MED15 promoter region.

Bibliographic details of certain publications numerically referred to in this specification are collected at the end of the description.

### BACKGROUND OF THE INVENTION

A carcinoma is a tumor tissue derived from putative epithelial cells that have become malignant. They invade surrounding tissues and organs and have the capacity to metastasize to other areas of the body. In their pre-malignant stage, they are sometimes referred to as “carcinoma in situ”, which have the cytological appearance of a malignant carcinoma but show no signs of invasion through the epithelial basement membrane.

Carcinomas are typically characterized by their histological appearance or their presumptive organ of origin. Examples include adenocarcinoma (e.g., renal cell carcinoma, hepatocellular carcinoma), squamous cell carcinoma (e.g., head and neck squamous cell carcinoma), adenosquamous carcinoma and basal cell carcinoma.

Carcinomas represent a substantial health and economic burden to society. For example, it is estimated that there are more than 900,000 cases of head and neck squamous cell carcinoma (HNSCC) diagnosed each year (1), accompanied by nearly 300,000 deaths (2). The primary risk factors for the development of HNSCC include tobacco use, alcohol consumption, human papillomavirus (HPV) infection (for oropharyngeal cancer) and Epstein-Barr virus (EBV) infections (for nasopharyngeal cancer) (3). In addition, betel nut chewing, which is common in certain regions of Asia, is also an independent risk factor for the development of HNSCC (4). The relative prevalence of these risk factors contributes to variations in the observed distribution of HNSCC in different parts of the world. As an example, oral and tongue cancers are common in the Indian subcontinent, nasopharyngeal cancers are common in China and Hong Kong, and pharyngeal and/or laryngeal cancers are prevalent in other populations (3).

Current diagnosis of carcinoma relies heavily on the histological assessment of tissue biopsy samples, tumor size, anatomic location and the presence of lymph node metastases. However, despite advances in knowledge and treatment of carcinomas such as HSCC, the survival rate of patients remains poor. For instance, the five-year survival rate for smoking-associated HNSCC is approximately 50% or less. This relatively high degree of mortality is largely attributed to late stage diagnosis, at which stage the malignant cells from the primary tumor have already metastasized. Hence, there is a clear need for the development of new methods for the detection of carcinoma in a subject that could lead to earlier diagnosis and an improvement of the survival rate of patients afflicted with this debilitating disease.

### SUMMARY OF THE INVENTION

Accordingly, in one aspect, the present invention provides a method for detecting the presence of a carcinoma or an increased likelihood that a carcinoma is present in a subject, the method comprising analyzing the DNA methylation status of the MED15 promoter in a biological sample obtained from the subject, and determining the presence of the carcinoma or increased likelihood that a carcinoma is present in the subject based on the analysis. Suitably, the presence of the carcinoma or increased likelihood that a carcinoma is present in the subject is determined when the analysis identifies that the DNA methylation status of the MED15 promoter is aberrant (e.g., hypermethylation). In specific embodiments, the carcinoma is a squamous cell carcinoma (e.g., head and neck squamous cell carcinoma).

Another aspect of the present invention provides a method of treating a carcinoma in a subject, the method comprising:

(a) analyzing the DNA methylation status of the MED15 promoter in a biological sample obtained from the subject;

(b) determining the presence of the carcinoma in the subject or an increased likelihood that a carcinoma is present in the subject based on the analysis; and

(c) exposing the subject to a treatment regimen for treating the carcinoma.

In a related aspect, the present invention provides a method of treating a carcinoma in a subject, the method comprising:

(a) sending a biological sample obtained from the subject to a laboratory to have an assay conducted, wherein the assay comprises analyzing the DNA methylation status of the MED15 promoter in the biological sample; and determining the presence of the carcinoma in the subject or an increased likelihood that a carcinoma is present in the subject based on the analysis;

(b) receiving the results of the assay of step (a); and

(c) exposing the subject to a treatment regimen for treating the carcinoma if the results indicate that the subject has or has an increased likelihood of having a carcinoma.

In another aspect of the present invention, there is provided a method for monitoring efficacy of a treatment regimen in a subject with a carcinoma, the method comprising:

(a) analyzing the DNA methylation status of the MED15 promoter in a biological sample obtained from the subject; and

(b) monitoring the subject over a period of time for a change in the methylation status of the MED15 promoter region;

wherein a change or otherwise in the methylation status of the MED15 promoter over the period of time is indicative of treatment efficacy.

Yet another aspect of the present invention provides a method for evaluating whether a subject is responding (i.e., a positive response) or not responding (i.e., a negative response or a lack of a positive response) to a treatment regimen for treating a carcinoma, the method comprising:

(a) analyzing the DNA methylation status of the MED15 promoter in a biological sample obtained from the subject following commencement of the treatment regimen; and

(b) correlating the DNA methylation status with a positive and/or negative response to the treatment regimen.

In yet another aspect, the present invention provides a method for determining a positive and/or negative response to a treatment regimen by a subject with a carcinoma, the method comprising:

(a) correlating DNA methylation status of the MED15 promoter with a positive or negative response to the treatment regimen to provide a correlated DNA methylation status;

(b) analyzing the DNA methylation status of the MED15 promoter in a biological sample obtained from the subject to provide a sample DNA methylation status, and

(c) determining whether the subject is responding to the treatment regimen based on the sample DNA methylation status and the correlated DNA methylation status.

In another aspect of the preset invention, there is provided a kit for detecting the presence of a carcinoma or an increased likelihood that a carcinoma is present in a subject, or for monitoring efficacy of a treatment regimen in a subject with a carcinoma, or for evaluating whether a subject is responding or not responding to a treatment regimen for treating a carcinoma, or for determining a positive and/or negative response to a treatment regimen by a subject with a carcinoma, suitably using the methods described herein, the kit comprising at least one agent for detecting the DNA methylation status of the MED15 promoter region.

In still another aspect, a method is provided for detecting the presence of a carcinoma or an increased likelihood that a carcinoma is present in a subject, the method comprising analyzing the DNA methylation status of the MED15 promoter and of at least one other promoter (suitably two or all three promoters) selected from the group consisting of p16^{INK4α}, RASSF1α and TIMP3 promoters in a biological sample obtained from the subject, and determining the presence of the carcinoma or an increased likelihood that a carcinoma is present in the subject based on the analysis.

A further aspect of the present invention provides a method of screening for the presence of a carcinoma or an increased likelihood that a carcinoma is present in a smoker (e.g., a tobacco user), the method comprising analyzing the DNA methylation status of the MED15 promoter and of at least one other promoter (suitably two or all three promoters) selected from the group consisting of p16^{INK 4α}, RASSF1α and TIMP3 promoters in a biological sample obtained from the smoker, and determining the presence of the carcinoma or an increased likelihood that a carcinoma is present in the smoker based on the analysis.

### BRIEF DESCRIPTION OF THE DRAWINGS

FIG. 1. Panel A is a schematic showing location of the sites in the main promoter-associated CpG island of the MED15/PCQAP promoter region. Panel B is the sequence (SEQ ID NOS: 12-25) of the two regions in the MED15/PCQAP promoter illustrating tumor-associated hypermethylation at the two CpG clusters (3′ and 5′) present in normal tissue and tumor samples obtained from tested subjects. The 5′ CpG cluster is located at positions 20,861,915 to 20,861,918 of human chromosome 22, with the differentially methylated cytosines representing the first and last residues of interval Chr22:20,861,915-20,861,918. The 3′ CpG cluster is located at positions 20,862,088 to 20,862,092 of human chromosome 22, with the differentially methylated cytosines representing the first and last residues of interval Chr22:20,862,088-20,862,092 (reference genome: GRCh37; GCA_000001405.13; Ensembl database; ensemble dot org). Results are shown for 3 patients (P1-P3). The reference sequence assumes full methylation at the CG dinucleotides.

FIG. 2. Panel A and Panel B are photomicrographs showing the methylation status of the two novel CpG sites using methylation specific polymerase chain reaction (MSP). Results for the upstream 5′ CpG cluster are shown in Panel A. Results for the downstream 3′ CpG cluster are shown in Panel B. MSP amplicons were separated by agarose gel electrophoresis, illustrating the higher level of methylation detectable in the saliva of HNSCC patients (see left 6 lanes, after the size markers) as compared to the level of methylation detectable in the saliva of healthy controls (next 6 lanes). No-template (NTC) and highly-methylated (HeLa) PCR controls are shown on the far right. DNA loading control (-Unmeth) and methylated target CpG-specific MSP amplicons (-Meth) for each patient are shown side-by-side.

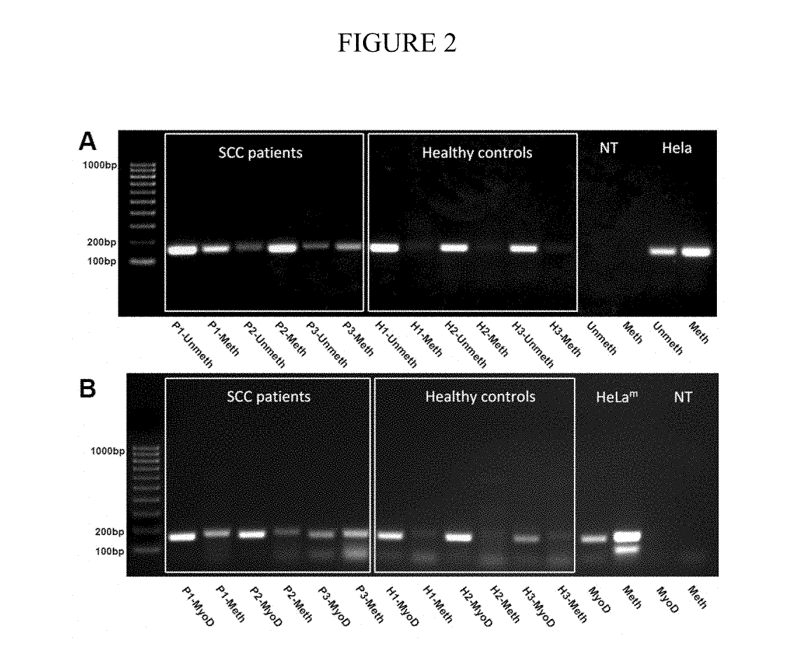

FIG. 3. Panel A and Panel B are scatter dot-plots showing the distribution of the relative methylation levels (i.e., the ratio of methylated to unmethylated forms) of the upstream 5′ CpG cluster (A) and the downstream 3′ CpG cluster (B) in control and HNSCC sample groups. Mann-Whitney test's results are shown (*** p<0.001; ** p<0.01).

FIG. 4. Panel A and Panel B are ROC curves for the 5′ (A) and 3′ (B) CpG cluster MSP analyses. AUC—area under the curve value.

FIG. 5 is a series of photomicrographs showing amplification of the methylated (-Meth) and/or unmethylated (-Unmeth) 3′ and 5′ CpG clusters by MSP from converted gDNA obtained from formalin-fixed, paraffin-embedded sections of HSCC tissue. It is to be noted that no methylated 3′MSP amplicons could be detected, while the level of detectable 5′MSP amplicons varied between patients.

FIG. 6 is a ROC curve for a 5-marker MSP analysis based on a comparison between healthy control smokers and HNSCC patients. The sensitivity and selectivity using logistic regression are 95% and 90%, respectively. This is applicable as a screen test.

FIG. 7 is a ROC curve for a 5-marker MSP analysis based on a comparison between healthy control non-smokers and HNSCC patients. The sensitivity and selectivity using logistic regression are 90% and 90%, respectively. This is applicable as a diagnostic test.

TABLE 1
BRIEF DESCRIPTION OF THE SEQUENCES

| | | |
|---|---|---|
| | | PCR |
| | | Product |
| | | Size |
| Gene | Nucleotide Sequence | (bp) |

Primer Sequences for CpG PCRs
(methylation-independent)

| | | |
|---|---|---|
| MED15_CpG- | Forward | 724 |
| Tag | 5′-CCA CTC ACT CAC CCA CCC GTA | |
| | GAA AAT GTA GG-3′ | |
| | [SEQ ID NO: 1] | |
| | Reverse | |
| | 5′-GGG TGG GAG GTG GGA GGG AAC | |
| | ACA CAA ATA AC-3′ | |
| | [SEQ ID NO: 2] | |
| Tag | Forward | |
| sequencing | 5′-CCA CTC ACT CAC CCA CCC-3′ | |
| | [SEQ ID NO: 3] | |
| | Reverse | |
| | 5′-GGG TGG GAG GTG GGA GGG-3′ | |
| | [SEQ ID NO: 4] | |

Primer Sequences for MSP

| | | |
|---|---|---|
| MED15_MSP5′ | Reverse | 167 |
| | 5′-AAA AAT CCC ACA ATC CAA | |
| | CCC-3′ | |
| | [SEQ ID NO: 5] | |
| | Forward Unmethylated | |
| | 5′-GTT TTG TGA TTG AGG YGG | |
| | TGG T-3′ | |
| | [SEQ ID NO: 6] | |
| | Forward Methylated | |
| | 5′-GTT TTG TGA TTG AGG YGG | |
| | CGG C-3′ | |
| | [SEQ ID NO: 7] | |
| MED15_MSP3′ | Forward | 172 |
| | 5′-GAT ATG GGT GGT GGG AGT | |
| | TGG G-3′ | |
| | [SEQ ID NO: 8] | |
| | Reverse Methylated | |
| | 5′-AAT CAG ACC CTA ACC TCG | |
| | CCC G-3′ | |
| | [SEQ ID NO: 9] | |
| MyoD | Forward | 158 |
| | 5′-TGA TTA ATT TAG ATT GGG | |
| | TTT AGA GAA-3′ | |
| | [SEQ ID NO: 10] | |
| | Reverse | |
| | 5′-CCA ACT CCA AAT CCC CTC | |
| | TCT AT-3′ | |
| | [SEQ ID NO: 11] | |

### DETAILED DESCRIPTION OF THE INVENTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present.

### 1. Abbreviations

The following abbreviations are used throughout the application:

nt=nucleotide

nts=nucleotides

aa=amino acid(s)

kb=kilobase(s) or kilobase pair(s)

kDa=kilodalton(s)

d=Day

h=hour

min=minute(s)

s=second(s)

### 2. Method of Diagnosis

The present invention is predicated in part on the determination that the promoter of the MED15 gene is differentially methylated in a biological sample obtained from a patient with a carcinoma as compared to a biological sample obtained from a normal subject or from a non-cancerous tissue sample obtained from the same subject. This finding allows the DNA methylation status of the MED15 promoter to be used as a diagnostic tool or epigenetic marker for detecting or predicting the presence of a carcinoma or an increased likelihood that a carcinoma is present in a subject.

Thus, in one aspect of the present invention, there is provided a method for detecting the presence of a carcinoma or an increased likelihood that a carcinoma is present in a subject, the method comprising analyzing the DNA methylation status of the MED15 promoter in a biological sample obtained from the subject, and determining the presence of the carcinoma or an increased likelihood that a carcinoma is present in the subject based on the analysis.

In some embodiments disclosed herein, the DNA methylation status of the MED15 promoter, or a segment thereof, is deemed to be hypermethylated (e.g., increased methylation as compared to the level of methylation of the MED15 promoter of a non-cancerous cell) when more than about 50%, 45%, 40%, 35%, 30%, 25%, 20%, 15%, 10%, 5%, 2%, or 1% methylation of the analyzed part of the MED15 promoter is detected as compared to, for example, the level of methylation of the MED15 promoter of a non-cancerous cell.

In some embodiments disclosed herein, the presence of the carcinoma or an increased likelihood that a carcinoma is present in the subject is based on increased methylation of the MED15 promoter when compared to the level of methylation of the MED15 promoter in a non-cancerous cell from the same subject. The non-cancerous cell may be a cell obtained from another organ not affected by the carcinoma (i.e., a healthy cell) or it may a normal (healthy) cell obtained from an area immediately adjacent the carcinoma. In some embodiments, the non-cancerous cell is a population of cells from the same subject or individual. In another embodiment, the non-cancerous cell is obtained from a one or more healthy individuals who do not have a carcinoma.

The terms “subject”, “individual” and “patient” are used interchangeably herein to refer to any subject, particularly a vertebrate subject, and even more particularly a mammalian subject. Suitable vertebrate animals that fall within the scope of the invention include, but are not restricted to, any member of the subphylum Chordata including primates, rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (e.g., snakes, frogs, lizards, etc.), and fish. A preferred subject is a primate (e.g., a human, ape, monkey, chimpanzee).

The term “carcinoma” would be understood by persons skilled in the art as tumor comprising cells derived from putative epithelial cells that have become malignant. The term also encompasses a carcinoma in situ, which is often used to describe a carcinoma in its pre-malignant stage; that is, having the cytological appearance of a malignant carcinoma but showing no signs of invasion through the epithelial basement membrane.

Carcinomas are typically characterized by their histological appearance or their presumptive organ of origin. Persons skilled in the art would be familiar with the different types of carcinoma. Examples include adenocarcinoma (e.g., renal cell carcinoma, hepatocellular carcinoma), squamous cell carcinoma (e.g., head and neck squamous cell carcinoma), adenosquamous carcinoma and basal cell carcinoma. The term carcinoma also refers to metastases derived from the primary tumor; that is, cells that have metastasized to other areas of the body, including those that may be found the circulation (e.g., circulating within blood vessels or the lymphatics). Thus, reference to a carcinoma is to be understood as a reference to the primary tumor and any metastases.

In some embodiments disclosed herein, the carcinoma is a squamous cell carcinoma. In non-limiting examples of this type, the carcinoma is a head and neck squamous cell carcinoma.

MED15 (mediator subunit complex 15) is a gene located on human chromosome 22q11 that encodes a pleiotropically-acting co-factor that is important for the assembly of the RNA polymerase II complex. The MED15 gene, also known as ARC105, CAG7A, CTG7A, PCQAP, TIG-1, TIG1, TNRC7, is responsible for the expression of all protein-coding genes (5). It possesses clearly-identifiable CpG islands associated with its main upstream promoter located between positions 20,861,680 to 20,862,252 of human chromosome 22 (GRCh37/hg19), comprising 54 CpG dinucleotides.

The term “gene” as used herein refers to any and all discrete coding regions of the cell's genome, as well as associated non-coding and regulatory regions of a DNA sequence. The term “gene” is also intended to mean the open reading frame encoding specific polypeptides, introns, and adjacent 5′ and 3′ non-coding nucleotide sequences involved in the regulation of expression. In this regard, the gene may further comprise control signals such as promoters, enhancers, termination and/or polyadenylation sites that are naturally associated with a given gene, or heterologous control signals.

The term “promoter” refers to a nucleic acid sequence, typically a region of a gene, that does not code for a protein, and that is operably linked or operably associated to a protein coding or RNA coding nucleic acid sequence such that the transcription of the operably linked or operably associated protein coding or RNA coding nucleic acid sequence is controlled by the promoter. Typically, eukaryotic promoters comprise between 100 and 5,000 base pairs, although this length range is not meant to be limiting with respect to the term “promoter” as used herein. Although typically found 5′ to the protein or RNA coding nucleic acid sequence to which they are operably linked or operably associated, promoters can be found in intron sequences as well. The term “promoter” is meant to include regulatory sequences operably linked or operably associated with the same protein or RNA encoding sequence that is operably linked or operably associated with the promoter. Promoters can comprise many elements, including regulatory elements. The term “promoter” comprises promoters that are inducible, wherein the transcription of the operably linked nucleic acid sequence encoding the protein is increased in response to an inducing agent. The term “promoter” may also comprise promoters that are constitutive, or not regulated by an inducing agent.

The term “DNA methylation status”, used herein to describe the state of methylation of a DNA sequence, including a genomic DNA sequence, refers to the characteristics of a DNA segment at a particular genomic locus relevant to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles. The term “DNA methylation status” also refers to the relative or absolute concentration of methylated C or unmethylated C at any particular stretch of residues in a biological sample. For example, if cytosine (C) residue(s) not typically methylated within a DNA sequence are methylated, it may be referred to as “hypermethylated”; whereas if cytosine (C) residue(s) typically methylated within a DNA sequence are not methylated, it may be referred to as “hypomethylated”. Likewise, if the cytosine (C) residue(s) within a DNA sequence (e.g., sample nucleic acid) are methylated as compared to another sequence from a different region or from a different individual (e.g., relative to normal nucleic acid), that sequence is considered hypermethylated compared to the other sequence. Alternatively, if the cytosine (C) residue(s) within a DNA sequence are not methylated as compared to another sequence from a different region or from a different individual, that sequence is considered hypomethylated compared to the other sequence. These sequences are said to be “differentially methylated”, and more specifically, when the DNA methylation status differs between a carcinoma and normal epithelial or non-tumor cells, the sequences are considered “differentially methylated between the carcinoma and normal epithelial or non-tumor cells”. Measurement of the levels of differential methylation may be done by a variety of ways known to those skilled in the art. One method is to measure the ratio of methylated to unmethylated alleles or (3-value. In some embodiments, the ratio of methylated to unmethylated alleles is measured by quantifying the amount of methylated and unmethylated forms of the DNA sequence of interest (e.g., by methylation-specific polymerase chain reaction (MSP), as described, for example, herein) and calculating the ratio of the quantity of methylated and unmethylated forms of the DNA. Thus, a fully unmethylated DNA sequence (i.e., having no detectable methylation) will have a ratio of 0.00. In non-limiting embodiments, the ratio of methylated to unmethylated regions in the MED15 promoter of DNA from a biological sample (e.g., saliva) of an HNSCC patient is from about 0.1 to about 15 (e.g., about 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 2.0, 3.0, 4.0, 5.0, 6.0. 7.0, 8.0, 9.0, 10.0, 11.0, 12.0, 13.0, 14.0 or about 15). In non-limiting embodiments, the ratio of methylated to unmethylated regions in the MED15 promoter of DNA from a biological sample (e.g., saliva) of an HNSCC patient is between 0.4 and 5.0, or between 0.5 and 5.0. In non-limiting embodiments, the ratio of methylated to unmethylated regions in the MED15 promoter of DNA from a biological sample (e.g., saliva) of a control subject (e.g., a subject without a carcinoma) is from about 0 to about 2 (e.g., about 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.5 or about 2.0). In non-limiting embodiments, the ratio of methylated to unmethylated regions in the MED15 promoter of DNA from a biological sample (e.g., saliva) of a control subject is between 0.00 and 0.4, or between 0.00 and 0.2. In other embodiments, a β-value can be used. β-values typically represent the normalized ratio between methylated and unmethylated alleles of a target DNA sequence and may vary between 0 (fully unmethylated) and 1 (fully methylated). In specific embodiments, the presence of a carcinoma or an increased likelihood that a carcinoma is present in the subject is indicated when the MED15 promoter in the biological sample obtained from the subject is hypermethylated as compared to the MED15 promoter in a corresponding biological sample obtained from a normal subject or from a subject lacking the carcinoma.

Analysis of methylation status may be performed through any suitable means known to persons skilled in the art. Several methylation analysis assays are known in the art, which may be used to practice the present invention. These assays allow for determination of the methylation status of one or a plurality of CpG sites within a nucleic acid sample. Non-limiting examples of methylation analysis assays include bisulfite genomic sequencing, methylation specific polymerase chain reaction (MSP), melting curve methylation-specific PCR (McMS-PCR), multiplex ligation-dependent probe amplification (MLPA) with or without bisulfite treatment, digestion of genomic DNA with methylation-sensitive restriction enzyme, multiplexed PCR with gene specific primers (MSRE-PCR; see (9)), bisulfite conversion-specific methylation-specific PCR (BS-MSP), methylation-sensitive single-nucleotide primer extension conformation (MS-SNuPE), methylation-sensitive single-strand conformation analysis (MS-SSCA), melting curve combined bisulfite restriction analysis (McCOBRA), enzymatic regional methylation assay (ERMA), quantitative PCR sequencing and oligonucleotide-based microarray systems, pyrosequencing, and Meth-DOP-PCR, a combination between a modified degenerate oligonucleotide primed PCR (DOP-PCR) and methylation-specific PCR (MSP). A review of some useful techniques for analyzing DNA methylation is provided by Laird P W (6).

Additionally, or alternatively, the identification of methylated nucleotides may also utilize the ability of the methyl binding domain (MBD) of the MeCP2 protein to selectively bind to methylated DNA sequences. The MBD may also be obtained from MBP, MBP2, MBP4, poly-MBD or from reagents such as antibodies binding to methylated nucleic acid. The MBD may be immobilized to a solid matrix and used for preparative column chromatography to isolate highly methylated DNA sequences. Variant forms such as expressed His-tagged methyl-CpG binding domain may be used to selectively bind to methylated DNA sequences. Other methods are well known in the art and include amongst others methylated-CpG island recovery assay (MIRA). Another method, MB-PCR, uses a recombinant, bivalent methyl-CpG-binding polypeptide immobilized on the walls of a PCR vessel to capture methylated DNA and the subsequent detection of bound methylated DNA by PCR.

In some embodiments, the method of analyzing the DNA methylation status of a nucleic acid of interest (e.g., a gene or region of a gene) is methylation specific PCR (MSP). MSP allows for assessing the methylation status of virtually any group of CpG sites within a CpG island, independent of the use of methylation-sensitive restriction enzymes (see, e.g., U.S. Pat. Nos. 5,786,146, 6,017,704, 6,200,756, 6,265,171 and US patent publication no. 2010/0144836). Briefly, DNA is modified by sodium bisulfite converting unmethylated, but not methylated cytosines to uracil, and subsequently amplified with primers specific for methylated versus unmethylated DNA. In non-limiting examples of the MSP approach, DNA is amplified using primer pairs designed to distinguish methylated from unmethylated DNA by taking advantage of sequence differences as a result of bisulfite or hydrazine ion treatment (see, e.g., (10)). For example, when sodium bisulfite is contacted to DNA, unmethylated cytosine is converted to uracil, while methylated cytosine is not modified. Uracil bases hybridize to adenine bases under hybridization conditions. Thus, an oligonucleotide primer which comprises adenine bases in place of guanine bases would hybridize to the bisulfite-modified DNA, whereas an oligonucleotide primer containing guanine bases would hybridize to the non-modified (methylated) cytosine residues in the DNA. Amplification using a DNA polymerase and a second primer yield amplification products (amplicons) that can be readily observed, which in turn indicates whether the DNA was methylated or not. The amplicons may be assessed directly using methods well known in the art. For example, amplicons may be visualized on a suitable gel, such as an agarose or polyacrylamide gel. Detection may involve the binding of specific dyes, such as ethidium bromide, which intercalate into double-stranded DNA and visualization of the DNA bands under a UV illuminator for example. Another means for detecting amplicons comprises hybridization with oligonucleotide probes. Alternatively, fluorescence or energy transfer can be measured to determine the presence of the methylated DNA.

In some embodiments disclosed herein, DNA is modified by treatment with sodium bisulfite, converting the unmethylated, but not methylated, cytosines to uracil. A subsequent amplification is performed with primers that are specific for methylated versus unmethylated DNA (7).

Variations on MSP, such as the use of nested and/or multiplex PCR, are also included within the scope of the present invention.

A specific example of the MSP technique is designated real-time quantitative MSP (QMSP), which permits quantification of methylated DNA in real time or at end point. Real-time methods are generally based on the continuous optical monitoring of an amplification procedure and utilize fluorescently labeled reagents whose incorporation in a product can be quantified and whose quantification is indicative of copy number of that sequence in the template. One such reagent is a fluorescent dye, called SYBR Green I that preferentially binds double-stranded DNA and whose fluorescence is greatly enhanced by binding of double-stranded DNA. Alternatively, labeled primers and/or labeled probes can be used for quantification. They represent a specific application of the well-known and commercially available real-time amplification techniques. In real-time PCR systems, it is possible to monitor the PCR reaction during the exponential phase where the first significant increase in the amount of PCR product correlates to the initial amount of target template. Real-Time PCR detects the accumulation of amplicon during the reaction. Where real-time PCR is used, quantitation may be on an absolute basis, or may be relative to a constitutively methylated DNA standard, or may be relative to an unmethylated DNA standard.

Methylation status may be determined by using the ratio between the signal of the marker under investigation and the signal of a reference nucleic acid where methylation status is known (such as Myosin D gene), or by using the ratio between the methylated marker and the sum of the methylated and the non-methylated marker. Alternatively, absolute copy number of the methylated marker can be determined.

Techniques that utilize restriction endonucleases to analyze the DNA methylation status of a nucleic acid of interest would be known to persons skilled in the art. Endonucleases may either preferentially cleave methylated recognition sites relative to non-methylated recognition sites or preferentially cleave non-methylated relative to methylated recognition sites. Some examples of the former are AccIII, BanI, BstNI, MspI, and XmaI. Examples of the latter are AccII, AvaI, BssHII, BstUI, HpaII, and NotI. Differences in cleavage pattern are indicative for the presence or absence of a methylated CpG dinucleotide. Cleavage patterns can be detected directly, or after a further reaction which creates products which are easily distinguishable. Means which detect altered size and/or charge can be used to detect modified products, including but not limited to electrophoresis, chromatography, and mass spectrometry.

In some embodiments, restriction enzyme digestion of PCR products amplified from bisulfite-converted DNA can be used to detect DNA methylation. By using methylation-sensitive or methylation-dependent restriction enzyme under conditions that allow for at least some copies of potential restriction enzyme cleavage sites in the locus to remain uncleaved, and subsequently quantifying the remaining intact copies and comparing the quantity to a control, the average methylation density of a locus can be determined. If the methylation-sensitive restriction enzyme is contacted to copies of a DNA locus under conditions that allow for at least some copies of potential restriction enzyme cleavage sites in the locus to remain uncleaved, then the remaining intact DNA will be directly proportional to the methylation density, and thus may be compared to a control to determine the relative methylation density of the locus in the sample. Similarly, if a methylation-dependent restriction enzyme is contacted to copies of a DNA locus under conditions that allow for at least some copies of potential restriction enzyme cleavage sites in the locus to remain uncleaved, then the remaining intact DNA will be inversely proportional to the methylation density, and thus may be compared to a control to determine the relative methylation density of the locus in the sample.

Other examples of methods for analyzing methylated DNA sequences use chemical reagents that selectively modify either the methylated or non-methylated form of CpG dinucleotide motifs. Suitable chemical reagents include hydrazine and bisulfite ions. In an embodiment disclosed herein, the method of the present invention utilizes bisulfite treatment. As hereinbefore described, bisulfite conversion relies on treatment of DNA samples with sodium bisulfite, which converts unmethylated cytosine to uracil, while methylated cytosines are maintained. This conversion results in a change in the sequence of the original DNA. It is general knowledge that the resulting uracil has the base pairing behavior of thymidine which differs from cytosine base pairing behavior. This makes the discrimination between methylated and non-methylated cytosines possible. Useful conventional techniques of molecular biology and nucleic acid chemistry for assessing sequence differences are well known in the art and explained in the literature (see, e.g., (11)).

Other suitable techniques known to persons skilled in the art use sequence specific primers for analyzing the methylation status of a gene of interest. Primers may be designed so that they themselves do not cover any potential sites of DNA methylation. Sequence variations at sites of differential methylation are located between the two primers and visualization of the sequence variation requires further assay steps. Alternatively, primers may be designed that hybridize specifically with either the methylated or unmethylated version of the initial bisulfite treated DNA sequence. After hybridization, an amplification reaction can be performed and the amplicons assayed using any detection system known to persons skilled in the art. The presence of an amplicon indicates that a sample hybridized to the primer. The specificity of the primer indicates whether the DNA had been modified or not, which in turn indicates whether the DNA had been methylated or not. If there is a sufficient region of complementarity, e.g., 12, 15, 18, or 20 nucleotides, to the target sequence, then the primer may also contain additional nucleotide residues that do not interfere with hybridization but may be useful for other manipulations. Examples of such other residues may be sites for restriction endonuclease cleavage, for ligand binding or for factor binding or linkers or repeats. The oligonucleotide primers may or may not be such that they are specific for modified methylated residues.

In some embodiments disclosed herein, MSP primers are utilized. Examples of suitable primers useful for analyzing the methylation status of the MED15 promoter are set forth in Table 1.

It would be understood by persons skilled in the art that variants of sequence-specific primers may be utilized in accordance with the present invention. For example, additional flanking sequences may be added that may, for example, improve binding specificity, as required. Variant sequences may have at least 90%, at least 91%, at least 92%, at least 93%, at least 94%, at least 95%, at least 96%, at least 97%, at least 98%, or at least 99% nucleotide sequence identity with the nucleotide sequences of the primers and/or probes set forth herein. The primers and probes may incorporate synthetic nucleotide analogues as appropriate or may be DNA, RNA or PNA based for example, or mixtures thereof. Similarly alternative fluorescent donor and acceptor moieties/FRET pairs may be utilized as appropriate. In addition to being labeled with the fluorescent donor and acceptor moieties, the primers and probes may include modified oligonucleotides and other appending groups and labels provided that the functionality as a primer and/or probe in the methods of the invention is not compromised.

In other embodiments, the MethyLight and Heavy Methyl assays may be used, which are high-throughput quantitative methylation assays that utilize a fluorescence-based real-time PCR (e.g., TaqMan®) technology that requires no further manipulations after the PCR step (see, e.g., (13), (14) and U.S. Pat. No. 6,331,393. Briefly, the MethyLight process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed either in an “unbiased” (with primers that do not overlap known CpG methylation sites) PCR reaction, or in a “biased” (with PCR primers that overlap known CpG dinucleotides) reaction. Sequence discrimination can occur either at the level of the amplification process or at the level of the fluorescence detection process, or both. The MethyLight assay may be used as a quantitative test for methylation patterns in the genomic DNA sample, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the “MSP” technique), or with oligonucleotides covering potential methylation sites. Typical reagents (e.g., as might be found in a typical MethyLight-based kit) for MethyLight analysis may include, but are not limited to: PCR primers for specific gene (or methylation-altered DNA sequence or CpG island); TaqMan® probes; optimized PCR buffers and deoxynucleotides; and Taq polymerase.

Alternatively, the Heavy Methyl technique begins with bisulfite conversion of DNA and specific blockers are then used to prevent amplification of unmethylated DNA. Methylated genomic DNA does not bind the blockers and their sequences will be amplified. The amplified sequences are detected with a methylation specific probe. (15).

Methylation-sensitive high resolution melting (HRM) is another useful assay that may be used to assess methylation. Non-limiting assays of this type are disclosed by Wojdacz and Dobrovic (16), Wojdacz et al. (17), Balic et al. (18) and US patent publication no. 2009/0155791. A variety of commercially available real time PCR machines have HRM systems including the Roche LightCycler480, Corbett Research RotorGene6000, and the Applied Biosystems 7500. HRM may also be combined with other amplification techniques such as pyrosequencing as described for example by Candiloro et al. (19).

Suitable controls may need to be incorporated in order to ensure the method is working reliably. Suitable controls may include assessing the methylation status of a gene known to be methylated. This acts as a positive control to help to ensure that false negative results are not obtained. The gene may be one which is known to be methylated in the sample under investigation or it may have been artificially methylated, for example by using a suitable methyltransferase enzyme.

Suitable negative controls may also be employed, including analyzing the methylation status of a gene known to be unmethylated or a gene that has been artificially demethylated. This provides a negative control to ensure against false positive results.

Other suitable amplification techniques for analyzing the DNA methylation status of the gene of interest include Transcription Mediated Amplification (TMA), ligase chain reaction (LCR), selective amplification of target polynucleotide sequences, consensus sequence primed polymerase chain reaction, arbitrarily primed polymerase chain reaction, strand displacement amplification and nick displacement amplification.

It would be understood by persons skilled in the art that a single method may suffice for analyzing the DNA methylation status of the gene of interest in accordance with the method of the present invention. Alternatively, the method of the present invention may utilize a combination of any two or more methods, as described, for example, herein.

The methods of the present invention, particularly where only a small amount of DNA is available, may require the amplification of the DNA of interest before testing for methylation of any specific gene. Suitable methods would be known to persons skilled in the art. Examples of whole genome amplification and libraries generation for such amplification include Methylplex and Enzyplex technology (Rubicon Genomics), as described, for example, in WO2004/081225. Modified degenerate oligonucleotide-primed PCR amplification (DOP-PCR) can also be combined with MSP to provides another suitable method for specific detection of methylation in small amount of DNA. An initial amplification of the gene or genes of interest, which is non-methylation specific may be carried out prior to the methylation detection method itself.

In some embodiments disclosed herein, the DNA methylation status of a target nucleic acid (e.g., a gene or a region of a gene) is analyzed by randomly shearing or randomly fragmenting the genomic DNA, cutting the DNA with a methylation-dependent or methylation-sensitive restriction enzyme and subsequently selectively identifying and/or analyzing the cut or uncut DNA. Selective identification can include, for example, separating cut and uncut DNA (e.g., by size exclusion chromatography, agarose gel electrophoresis) and quantifying a sequence of interest that was cut or, alternatively, the sequence that was not cut.

In other embodiments, the method comprises amplifying intact DNA after restriction enzyme digestion, thereby only amplifying DNA that was not cleaved by the restriction enzyme in the area amplified. In some embodiments, amplification can be performed using primers that are gene specific. Alternatively, adaptors can be added to the ends of the randomly fragmented DNA, the DNA digested with a methylation-dependent or methylation-sensitive restriction enzyme, and the intact DNA amplified using primers that hybridize to the adaptor sequences. In some embodiments, a second step can be performed to determine the presence, absence or quantity of a particular gene in an amplified pool of DNA. In some embodiments, the DNA is amplified using quantitative real-time PCR (RT-PCR).

In other embodiments disclosed herein, the method comprises quantifying the average methylation density in a target sequence within a population of genomic DNA. For example, the method can comprise contacting genomic DNA with a methylation-dependent restriction enzyme or methylation-sensitive restriction enzyme under conditions that allow for at least some copies of potential restriction enzyme cleavage sites in the locus to remain uncleaved. Intact copies of the locus are then quantified, followed by a comparison of the quantity of amplified product to a control value representing the quantity of methylation of control DNA (e.g., from non-cancerous cells), thereby quantifying the average methylation density in the locus compared to the methylation density of the control DNA.

The quantity of methylation of a locus of DNA can also be determined by providing a sample of genomic DNA comprising the locus, cleaving the DNA with a restriction enzyme that is either methylation-sensitive or methylation-dependent, and then quantifying the amount of intact DNA or quantifying the amount of cut DNA at the DNA locus of interest. It will be understood that the amount of intact or cut DNA will depend on the initial amount of genomic DNA containing the locus, the amount of methylation in the locus, and the number (i.e., the fraction) of nucleotides in the locus that are methylated in the genomic DNA. The amount of methylation in a DNA locus can be determined by comparing the quantity of intact DNA or cut DNA to a control value representing the quantity of intact DNA or cut DNA in a similarly-treated DNA sample. The control value can represent a known or predicted number of methylated nucleotides. Alternatively, the control value can represent the quantity of intact or cut DNA from the same locus in another (e.g., normal, non-diseased) cell or a second locus.

In some embodiments disclosed herein, quantitative amplification methods can be used to quantify the amount of intact DNA within a locus flanked by amplification primers following restriction digestion (e.g., via quantitative PCR or quantitative linear amplification). Methods of quantitative amplification are disclosed, e.g., in U.S. Pat. Nos. 6,180,349 and 6,033,854.

In some embodiments disclosed herein, a Ms-SNuPE (Methylation-sensitive Single Nucleotide Primer Extension) reaction can be used, either alone or in combination with other methods to detect DNA methylation (see, e.g., (12)). The Ms-SNuPE technique is a quantitative method for assessing methylation differences at specific CpG sites based on bisulfite treatment of DNA, followed by single-nucleotide primer extension. Genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product (amplicon) is isolated and used as a template for methylation analysis at the target site of interest.

In some embodiments disclosed herein, the DNA extracted from the biological sample is preamplified before bisulfite conversion. In some embodiments, the extracted DNA is preamplified before bisulfite conversion using the Invitrogen Superscript III One-Step RT-PCR System with Platinum Taq. In some embodiments, the DNA isolated from the tissue sample is preamplified before bisulfite conversion using a TaqMan based assay. In some embodiments, the sodium bisulfite reaction is conducted using the Zymo EZ DNA Methylation-Gold Kit (Zymo Research) or the EpiTectPlus™ (Qiagen GmbH).

In some embodiments, the bisulfite converted DNA product is amplified (e.g., via polymerase chain reaction; PCR) using primer pairs that are designed to specifically hybridize to methylated or unmethylated target sequences. Methods for amplifying sequence specific DNA by PCR would be known to persons skilled in the art. Examples include commercial kits such as the Invitrogen Superscript III One-Step RT-PCR System with Platinum Taq or AmpliTaq Gold 360 Master Mix (Applied Biosystems, USA).

In some embodiments, the methylation status of DNA is determined by hybridization. For example, after sodium bisulfite treatment of DNA, oligonucleotides complementary to potential methylation sites can hybridize to the bisulfite-treated DNA. The oligonucleotides are designed to be complementary to either the sequence containing uracil (thymine) or the sequence containing cytosine, representing unmethylated and methylated DNA, respectively. Computer-based microarray technology can determine which oligonucleotides hybridize with the DNA sequence and from there one can deduce the methylation status of the DNA.

Another non-limiting example of a method for determining the presence of methylated nucleotides involves sequencing the bisulfite treated DNA to directly observe any bisulfite-modifications. Suitable sequencing methods would be known to persons skilled in the art. For example, pyrosequencing is a method of sequencing-by-synthesis in real time. It is based on an indirect bioluminometric assay of the pyrophosphate (PPi) that is released from each deoxynucleotide (dNTP) upon DNA-chain elongation. This method presents a DNA template-primer complex with a dNTP in the presence of an exonuclease-deficient Klenow DNA polymerase. The four nucleotides are sequentially added to the reaction mix in a predetermined order. If the nucleotide is complementary to the template base and thus incorporated, PPi is released. The PPi and other reagents are used as a substrate in a luciferase reaction producing visible light that is detected by either a luminometer or a charge-coupled device. The light produced is proportional to the number of nucleotides added to the DNA primer and results in a peak indicating the number and type of nucleotide present in the form of a pyrogram. Pyrosequencing can exploit the sequence differences that arise following sodium bisulfate-conversion of DNA.

The methylation status of the MED15 gene may be analyzed by determining the level of methylation in the MED15 promoter and, optionally, in one or more introns, in one or more exons, or combinations thereof. In some embodiments disclosed herein, the method comprises analyzing the DNA methylation status of the MED15 promoter. A promoter is typically found upstream from the transcription start site (TSS), extending between approximately 10 Kb, 4 Kb, 3 Kb, 1 Kb, 500 bp or 150 to 300 bp from the TSS. The nucleic acid region for assessment may be a region that comprises both intron and exon sequences and thus overlaps both regions.

The present inventors have also surprisingly found two novel CpG clusters in the promoter of the MED15 gene between positions 20,861,680 and 20,862,252 of human chromosome 22 that are hypermethylated in carcinoma, as compared to normal epithelial or non-tumor cells. “CpG” is shorthand for “-C-phosphate-G-”, that is, a series of cytosine and guanine residues separated by only a phosphate molecule. The “CpG” notation is used to distinguish this linear sequence from the CG base-pairing of cytosine and guanine. The term “CpG cluster” or “CpG site”, as used herein, means a region of DNA comprising a series of CpG dinucleotides. The term “CpG island”, as used herein, means a GC-rich region of DNA that comprises a high frequency of CpG clusters.

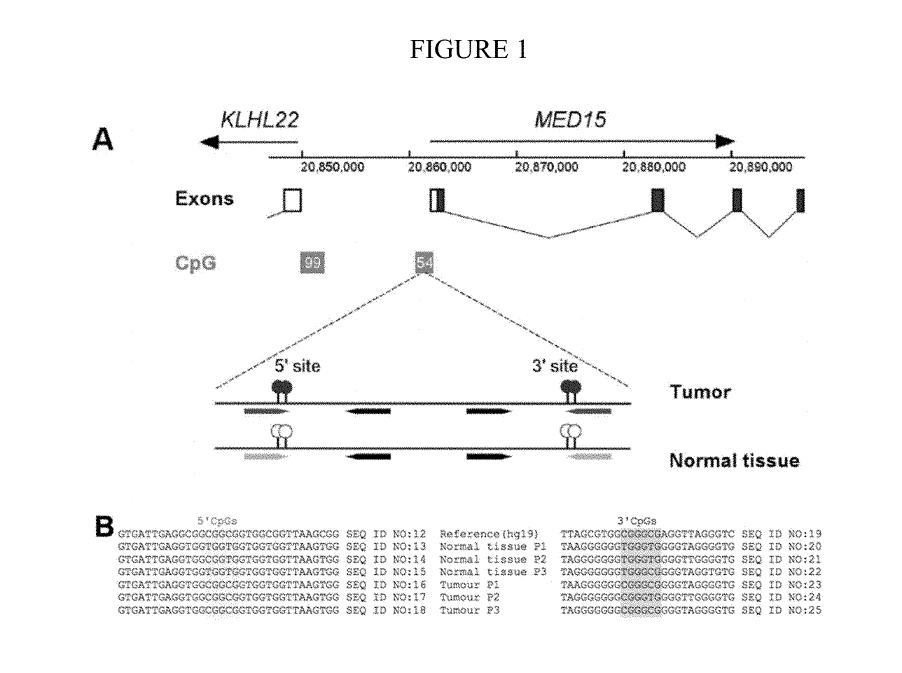

Thus, in some embodiments disclosed herein, the presence of carcinoma or an increased likelihood that a carcinoma is present in the subject is based on increased methylation of at least one CpG cluster of the MED15 promoter. In some embodiments, the CpG cluster is located at the 5′ end of the region defined by positions 20,861,680 to 20,862,252 of human chromosome 22. In a non-limiting embodiment, the 5′ CpG cluster is located at positions 20,861,915 to 20,861,918 of human chromosome 22 (reference genome: GRCh37; GCA_000001405.13; Ensembl database; ensembl dot org), wherein the differentially methylated cytosines are represented by the first and last residues of interval Chr22:20,861,915-20,861,918. Thus, in some embodiments, the presence of carcinoma or an increased likelihood that a carcinoma is present in the subject is based on increased methylation at the 5′ CpG cluster. In some embodiments, the CpG cluster is located at the 3′ end of the region defined by positions 20,861,680 to 20,862,252 of human chromosome 22. In a non-limiting embodiment, the 3′ CpG cluster is located at positions 20,862,088 to 20,862,092 of human chromosome 22 (reference genome: GRCh37; GCA_000001405.13; Ensembl database; ensembl dot org), wherein the differentially methylated cytosines are represented by the first and last residues of interval Chr22:20,862,088-20,862,092. Thus, in some embodiments, the presence of carcinoma or an increased likelihood that a carcinoma is present in the subject is based on increased methylation at the 3′ CpG cluster. In some embodiments, the presence of carcinoma or an increased likelihood that a carcinoma is present in the subject is based on increased methylation at both the 5′ and 3′ CpG clusters in the region defined by positions 20,861,680 to 20,862,252 of human chromosome 22, as described, e.g., herein. In non-limiting embodiments, the ratio of methylated to unmethylated forms of the 5′ CpG cluster of the MED15 promoter (as shown, e.g., in FIG. 1) of DNA from a biological sample (e.g., saliva) of an HNSCC patient is at least about 0.4 (e.g., about 0.45, 0.5, 0.55, 0.6, 0.65, 0.7, 0.75, 0.8, 0.85, 0.9, 0.95, 1.0, 2.0, 3.0, 4.0, 5.0, 6.0. 7.0, 8.0, 9.0, 10.0, 11.0, 12.0, 13.0, 14.0 or about 15.0). In non-limiting embodiments, the ratio of methylated to unmethylated forms of the 5′ CpG cluster of the MED15 promoter of DNA from a biological sample (e.g., saliva) of an HNSCC patient is between 0.5 and 15, or between 0.5 and 5.0. In non-limiting embodiments, the ratio of methylated to unmethylated forms of the 5′ CpG cluster of the MED15 promoter (as shown, e.g., in FIG. 1) of DNA from a biological sample (e.g., saliva) of a control subject (e.g., a subject without carcinoma) is less than about 0.4 (e.g., about 0.35, 0.3, 0.25, 0.2, 0.15, 0.1, 0.05 or 0.00). In non-limiting embodiments, the ratio of methylated to unmethylated forms of the 5′ CpG cluster of the MED15 promoter of DNA from a biological sample (e.g., saliva) of a control subject is between 0.00 and 0.3, or between 0.05 and 0.3. In non-limiting embodiments, the ratio of methylated to unmethylated forms of the 3′ CpG cluster of the MED15 promoter (as shown, e.g., in FIG. 1) of DNA from a biological sample (e.g., saliva) from an HNSCC patient is at least about 0.13 (e.g., about 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.5, 2.0, 2.5, 3.0, 3.5 or about 4.0). In non-limiting embodiments, the ratio of methylated to unmethylated forms of the 3′ CpG cluster of the MED15 promoter of DNA from a biological sample (e.g., saliva) from an HNSCC patient is between 0.13 and 4.0, or between 0.13 and 2.0. In non-limiting embodiments, the ratio of methylated to unmethylated forms of the 3′ CpG cluster of the MED15 promoter (as shown, e.g., in FIG. 1) of DNA from a biological sample (e.g., saliva) of a control subject is less than about 0.13 (e.g., about 0.1, 0.09, 0.08, 0.07, 0.06, 0.05, 0.04, 0.03, 0.02, 0.01 or 0.00). In non-limiting embodiments, the ratio of methylated to unmethylated forms of the 3′ CpG cluster of the MED15 promoter of DNA from a biological sample (e.g., saliva) of a control subject is between 0.00 and 0.12, or between 0.00 and 0.10.

Methods for analyzing the DNA methylation status of CpG clusters would be known to persons skilled in the art, such as those described, for example, herein (see also, e.g., (8)). In many genes, the CpG islands are found in the promoter and may begin (just) upstream of a promoter and extend downstream into the transcribed region. Methylation of a CpG island in a promoter often prevents expression of the gene. CpG islands can also surround the 5′ region of the coding region of a gene as well as the 3′ region of the coding region. Thus, CpG islands can be found in multiple regions of a nucleic acid sequence including upstream of coding sequences in a regulatory region including a promoter region, in the coding regions (e.g. exons), downstream of coding regions in, for example, enhancer regions, and in introns. All of these regions can be assessed to determine their methylation status, as appropriate. CpG islands and CpG clusters are readily identifiable through a range of techniques known to persons skilled in the art, including sequencing and in silico predictive methods.

The term “biological sample” as used herein refers to a sample that may be extracted, untreated, treated, diluted or concentrated from a subject. The biological sample can be any sample obtained from the subject that is reasonably expected to comprise nucleic acid (e.g., genomic DNA) of cells from a primary or secondary (e.g., metastatic) carcinoma, or from cells that are shed from a primary or secondary carcinoma and collected in biological fluids. Non-limiting examples of biological samples include, but are not limited to, tissue, bodily fluid (for example, blood, serum, plasma, saliva, urine, tears, peritoneal fluid, ascitic fluid, vaginal secretion, breast fluid, breast milk, lymph fluid, cerebrospinal fluid or mucosa secretion), umbilical cord blood, chorionic villi, amniotic fluid, an embryo, embryonic tissues, lymph fluid, cerebrospinal fluid, mucosa secretion, or other body exudate, fecal matter, an individual cell or extract of the such sources that contain the nucleic acid of the same, and subcellular structures such as mitochondria, using protocols well established within the art.

In some embodiments disclosed herein, the biological sample is a clinical sample obtained from a primary or metastatic tumor. For example, a tissue biopsy is often used to obtain a representative piece of tumor tissue. Alternatively, the biological sample can be obtained indirectly in the form of tissues or fluids that are known or thought to contain the tumor cells of interest or DNA therefrom. For instance, samples of lung cancer lesions may be obtained by resection, bronchoscopy, fine needle aspiration, bronchial brushings, or from sputum, saliva, pleural fluid or blood. In some embodiments, the sample includes circulating tumor cells; for example, circulating cancer cells in blood, lymph, urine or sputum.

In some embodiments disclosed herein, the biological sample is a bodily fluid or excretion such as blood, urine, saliva, stool, pleural fluid, lymphatic fluid, sputum, ascites, prostatic fluid, cerebrospinal fluid (CSF), or any other bodily secretion or extract thereof. Blood samples include whole blood, plasma, serum or extracts thereof. The analysis of DNA methylation in such biological fluids or excretions can sometimes be preferred, particularly in circumstances where an invasive sampling method is inappropriate or inconvenient.

In some embodiments disclosed herein, the biological sample comprises a lung cancer tumor cell (e.g. non-small cell lung cancer (NSCLC)), a pancreatic cancer tumor cell, a breast cancer tumor cell, a head and neck squamous cell carcinoma cell, a gastric cancer tumor cell, a colon cancer tumor cell, an ovarian cancer tumor cell, or a tumor cell from any of a variety of other carcinomas, as described, for example, herein.

In some embodiments disclosed herein, the biological sample is a tissue sample of the primary tumor. Such samples can be obtained by any means known to persons skilled in the art, e.g., via tissue biopsy, surgical resection or buccal cell scrape. In some embodiments disclosed herein, the biological sample is a buccal cell scrape.

The biological sample may be processed and analyzed in accordance with the methods of the present invention almost immediately following collection (i.e., as a fresh tissue sample), or it may be stored for subsequent analysis. If storage of the tissue sample is desired or required, it would be understood by persons skilled in the art that it should ideally be stored under conditions that preserve the integrity of the DNA within the tissue sample (e.g., at −80° C.). Thus, in some embodiments disclosed herein, the biological sample is a fresh frozen tissue sample. Tissue samples may also be stored as formalin-fixed paraffin embedded (FFPE) tissue, such as those prepared by pathologists for immunohistochemical analysis. Thus, in some embodiments disclosed herein, the biological sample is an FFPE tissue sample.

The present inventors have also determined that saliva is a suitable biological sample for the purposes of analyzing the DNA methylation status of a DNA sequence, including the MED15 promoter region, Thus, in some embodiments disclosed herein, the biological sample is saliva. Without being bound by theory, it is hypothesized that cells may be sloughed off from the carcinoma (primary or secondary tumors/metastases) and appear in biological samples such as saliva. By screening such samples, a simple, non-invasive method for the early detection of a carcinoma can be achieved. In addition, the progress of therapy can be monitored more easily by analyzing such biological samples for the DNA methylation status of the MED15 promoter in accordance with the present invention, as described, for example, herein.

In some embodiments, analysis of the DNA methylation status of the MED15 promoter may be performed on a biological sample prior to extracting DNA. However, it would be understood by persons skilled in the art that, where the method is performed using raw biological material (i.e., prior to DNA extraction) the conditions may need to be optimized so as to allow for the detection of DNA methylation of the target sequence. For example, a method may incorporate an agent in situ that lyses the cellular and/or nuclear membranes of the biological sample so as to allow the release of genomic DNA. Alternatively, no additional step may be required, particularly where the biological sample comprises a sufficient quantity of naked DNA that has, for example, been shed by a cell during storage or during removal from the subject.

In some embodiments disclosed herein, the biological sample is initially processed to extract DNA from the biological sample before DNA methylation analysis. Suitable methods of extracting DNA from a biological sample would be known to persons skilled in the art. Non-limiting examples include the use of commercial DNA extraction kits such as EpiTectPlus™ (Qiagen GmbH) in accordance with the manufacturers' instructions.

In some embodiments disclosed herein, the biological sample is a buccal cell scrape, or an extract thereof. In some embodiments disclosed herein, the biological sample is saliva, or an extract thereof.

Persons skilled in the art would understand that a combination of methods for analyzing the DNA methylation status of the MED15 promoter region, as described, for example, herein, may be employed with a view to improving the diagnostic capacity of the methods of the present invention.

Persons skilled in the art would also understand that that the analysis of multiple DNA methylation sites may augment efficient carcinoma identification in accordance with the method of the present invention. For example, additional genetic markers (i.e., other than the methylation status of the MED15 promoter) may be used.

The additional genetic markers may concern mutation markers that allow detection of mutations in distinct genes, or, alternatively epigenetic markers that allow detection of DNA methylation in other genes. Thus, the diagnostic potential of the method of the present invention may be improved by analyzing additional markers that are also predictive of the presence of the carcinoma or an increased likelihood that a carcinoma is present in the subject. Suitable markers would be known to persons skilled in the art. In some embodiments, additional markers include the respective methylation status of any one or more of the promoters of DAPK1, p16^{INK4α}, RASSF1α, as described, for example, by Ovchinnikov et al (7), and/or DCC, DAPK, TIMP3, ESR, CCNA1, CCND2,MINT1, MINT31, CDH1, AIM1, MGMT, p16, PGP9.5, RARB, HIC1, RASSF1A, CALCA, TGFBR2, S100A2, RIZ1, RBM6, KIF 1, EDNRB and TIMP3, as described for example in U.S. Publication No. 2011/0097724 and Sun et al. (20, 21). In specific embodiments, the or each additional marker is selected from the methylation status of the promoter of at least one or more (e.g., 1, 2 or 3) genes selected from DAPK1, p16^{INK4α} and RASSF1α. In other specific embodiments, the or each additional marker is selected from the methylation status of the promoter of at least one or more (e.g., 1, 2, 3 or 4) genes selected from DAPK1, p16^{INK4α}, RASSF1α and TIMP3.). In still other specific embodiments, the or each additional marker is selected from the methylation status of the promoter of at least one or more (e.g., 1, 2 or 3) genes selected from p16^{INK4α} RASSF 1α and TIMP3.

Particularly advantageous embodiments of the present invention employ the DNA methylation status of the MED15 promoter region in combination with the DNA methylation status of at least 1, 2 or all 3 biomarkers selected from the group consisting of p16^{INK4α}, RASSF1α and TIMP3 (preferably their promoter regions) to provide a biomarker panel that is useful not only for distinguishing between healthy individuals and HNSCC patients but also for distinguishing between non-HNSCC smokers (also referred to herein as “healthy smokers”) and HNSCC patients.

Accordingly, the present invention provides a method for detecting the presence of a carcinoma or an increased likelihood that a carcinoma is present in a subject, the method comprising analyzing the DNA methylation status of the MED15 promoter and of at least one other promoter selected from the group consisting of p16^{INK4α}, RASSF1α and TIMP3 promoters in a biological sample obtained from the subject, and determining the presence of the carcinoma or an increased likelihood that a carcinoma is present in the subject based on the analysis. In another aspect, the present invention also contemplates a method of screening (i.e., a screening test) for the presence of a carcinoma or an increased likelihood that a carcinoma is present in a smoker (e.g., a tobacco user), the method comprising analyzing the DNA methylation status of the MED15 promoter and of at least one other promoter selected from the group consisting of p16^{INK4α}, RASSF1α and TIMP3 promoters in a biological sample obtained from the smoker, and determining the presence of the carcinoma or an increased likelihood that a carcinoma is present in the smoker based on the analysis. In specific embodiments of the above aspects, the method comprises analyzing the DNA methylation status of the respective promoters of the MED15, p16^{INK4α}, RASSF1α and TIMP3 genes.

Thus, in some embodiments disclosed herein, the method of the present invention further comprises analyzing the DNA methylation status of at least one other marker (e.g., epigenetic marker, including a methylation epigenetic marker) associated with the presence of the carcinoma in a subject, or with an increased likelihood that the carcinoma is present in a biological sample obtained from the subject, and determining the presence of the carcinoma or an increased likelihood that a carcinoma is present in the subject based on the analysis of the MED15 promoter and the analysis of the at least one other marker. In illustrative examples of this type, the at least one other marker is selected from promoters of the DAPK1, p16^{INK4α} and RASSF1α genes. In other illustrative examples, the at least one other marker is selected from promoters of the DCC, DAPK, TIMP3, ESR, CCNA1, CCND2, MINT1, MINT31, CDH1, AIM1, MGMT, p16, PGP9.5, RARB, HIC1, RASSF1A, CALCA, TGFBR2, S100A2, RIZ1, RBM6, KIF1, EDNRB and TIMP3 genes. In specific examples, the at least one other marker is selected from promoters of the DAPK1, p16^{INK4α} RASSF1α and TIMP3 genes. In other specific examples, the at least one other marker is selected from promoters of the p16^{INK4α}, RASSF1α and TIMP3 genes. In certain embodiments, the method of the present invention further comprises analyzing the DNA methylation status of 2, 3 or 4 promoters selected from the group consisting of promoters of the DAPK1, p16^{INK4α}, RASSF1α and TIMP3 genes. In other embodiments, the method of the present invention further comprises analyzing the DNA methylation status of 2 or 3 promoters selected from the group consisting of promoters of the p16^{INK4α}, RASSF1α and TIMP3 genes.

As used herein, the term “epigenetic marker” refers to a nucleotide sequence that is differentially epigenetically modified in a carcinoma (e.g., a squamous carcinoma including head and neck squamous carcinoma), as compared to the nucleotide sequence in a normal or non-tumor or control cell. The epigenetic marker may be hypermethylated or hypomethylated in the disorder or disease state relative to the normal, non-tumor or control cell. In general, the epigenetic marker comprises between about 5 and about 10000 nucleotides, for example, but not limited to 5, 7, 9, 11, 15, 17, 21, 25, 50, 75, 100, 200, 300, 400, 500, 600, 700, 800, 900 or 1000, 2000, 3000, 4000, 5000, 6000, 7000, 8000, 9000, 10000 nucleotides, or any amount therein between. Further, the epigenetic marker may comprise a range of sizes as defined by any two of the values listed or any two amounts therein between.

In some embodiments, the presence of the carcinoma or an increased likelihood that a carcinoma is present in the subject is based on increased methylation of the MED15 promoter and increased methylation of the promoter of the one or more genes selected from the group consisting of DAPK1, p16^{INK4α} and RASSF1α when compared to the level of methylation of the same promoter in a non-cancerous cell from the same subject, as described, for example, herein.

In other embodiments, the presence of the carcinoma or an increased likelihood that a carcinoma is present in the subject is based on increased methylation of the MED15 promoter and increased methylation of the promoter of the 1, 2, 3 or 4 genes selected from the group consisting of DAPK1, p16^{INK4α}, RASSF1α and TIMP3 when compared to the level of methylation of the same promoter in a non-cancerous cell from the same subject, as described, for example, herein.

In still other embodiments, the presence of the carcinoma or an increased likelihood that a carcinoma is present in the subject is based on increased methylation of the MED15 promoter and increased methylation of the promoter of the 1, 2 or 3 genes selected from the group consisting of p16^{INK4α}, RASSF1α and TIMP3 when compared to the level of methylation of the same promoter in a non-cancerous cell from the same subject, as described, for example, herein.

In some embodiments, the methods comprise comparing the DNA methylation status of a nucleic acid of interest to a preselected or threshold DNA methylation status. Thresholds may be selected that provide an acceptable ability to predict diagnosis, likelihood, prognostic risk, treatment success, etc. As used herein, the term “likelihood” is used as a measure of whether subjects with a particular methylation status actually have a carcinoma (or not) based on a given mathematical model. An increased likelihood for example may be relative or absolute and may be expressed qualitatively or quantitatively. For instance, an increased risk may be expressed as simply determining the subject's methylation status of a nucleic acid of interest (e.g., the promoter of the MED15 gene, and optionally at least one other epigenetic marker) and placing the test subject in an “increased risk” category, based upon previous population studies. Alternatively, a numerical expression of a test subject's increased risk may be determined based upon an analysis of the subject's methylation status per se.

In illustrative examples, receiver operating characteristic (ROC) curves are calculated by plotting the value of a variable versus its relative frequency in two populations in which a first population has a first condition or risk and a second population has a second condition or risk (called arbitrarily, for example, “healthy condition” and “carcinoma”, “a first stage or severity of carcinoma” and “a second stage or severity of carcinoma”, or “low risk” and “high risk”).

A distribution of DNA methylation statuses for subjects with and without a disease will likely overlap. Under such conditions, a test does not absolutely distinguish a first condition and a second condition with 100% accuracy, and the area of overlap indicates where the test cannot distinguish the first condition and the second condition. A threshold is selected, above which (or below which, depending on how DNA methylation status changes with a specified condition or prognosis) the test is considered to be “positive” and below which the test is considered to be “negative.” The area under the ROC curve (AUC) provides the C-statistic, which is a measure of the probability that the perceived measurement will allow correct identification of a condition (see, e.g., Hanley et al., Radiology 143: 29-36 (1982). The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g., a healthy condition DNA methylation status and a carcinoma DNA methylation status). ROC curves are useful for plotting the performance of a particular feature (e.g., a DNA methylation status described herein and/or any item of additional biomedical information) in distinguishing or discriminating between two populations (e.g., cases having a carcinoma and controls without the carcinoma). Typically, the feature data across the entire population (e.g., the cases and controls) are sorted in ascending order based on the value of a single feature. Then, for each value for that feature, the true positive and false positive rates for the data are calculated. The sensitivity is determined by counting the number of cases above the value for that feature and then dividing by the total number of cases. The specificity is determined by counting the number of controls below the value for that feature and then dividing by the total number of controls. Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to produce a single value, and this single value can be plotted in a ROC curve. Additionally, any combination of multiple features (e.g., one or more other epigenetic markers), in which the combination derives a single output value, can be plotted in a ROC curve. These combinations of features may comprise a test. The ROC curve is the plot of the sensitivity of a test against the specificity of the test, where sensitivity is traditionally presented on the vertical axis and specificity is traditionally presented on the horizontal axis. Thus, “AUC ROC values” are equal to the probability that a classifier will rank a randomly chosen positive instance higher than a randomly chosen negative one. An AUC ROC value may be thought of as equivalent to the Mann-Whitney U test, which tests for the median difference between scores obtained in the two groups considered if the groups are of continuous data, or to the Wilcoxon test of ranks.

Alternatively, or in addition, thresholds may be established by obtaining an earlier DNA methylation status result from the same patient, to which later results may be compared. In these embodiments, the individual in effect acts as their own “control group.” In DNA methylation levels that increase with condition severity or prognostic risk, an increase over time in the same patient can indicate a worsening of the condition or a failure of a treatment regimen, while a decrease over time can indicate remission of the condition or success of a treatment regimen.

In some embodiments, a positive likelihood ratio, negative likelihood ratio, odds ratio, and/or AUC or receiver operating characteristic (ROC) values are used as a measure of a method's ability to predict risk or likelihood, or to diagnose a disease or condition. As used herein, the term “likelihood ratio” is the probability that a given test result would be observed in a subject with a condition of interest divided by the probability that that same result would be observed in a patient without the condition of interest. Thus, a positive likelihood ratio is the probability of a positive result observed in subjects with the specified condition divided by the probability of a positive results in subjects without the specified condition. A negative likelihood ratio is the probability of a negative result in subjects without the specified condition divided by the probability of a negative result in subjects with specified condition. As used herein, the term “probability” refers to the probability of class membership for a sample as determined by a given mathematical model and is construed to be equivalent likelihood in this context.

The term “odds ratio”, as used herein, refers to the ratio of the odds of an event occurring in one group (e.g., a healthy condition group) to the odds of it occurring in another group (e.g., a carcinoma group, or a group with particular stage or severity of carcinoma), or to a data-based estimate of that ratio.

In some embodiments, an epigenetic marker or panel of markers, including at least one epigenetic marker, is selected to discriminate between subjects with a first condition and subjects with a second condition with at least about 50%, 55% 60%, 65%, 70%, 75%, 80%, 85%, 90%, 95% accuracy or having a C-statistic of at least about 0.50, 0.55, 0.60, 0.65, 0.70, 0.75, 0.80, 0.85, 0.90, 0.95.

In the case of a positive likelihood ratio, a value of 1 indicates that a positive result is equally likely among subjects in both the “condition” and “control” groups; a value greater than 1 indicates that a positive result is more likely in the condition group; and a value less than 1 indicates that a positive result is more likely in the control group. In this context, “condition” is meant to refer to a group having one characteristic (e.g., the presence of a healthy condition, carcinoma, or a particular stage or severity of carcinoma) and “control” group lacking the same characteristic.

In the case of a negative likelihood ratio, a value of 1 indicates that a negative result is equally likely among subjects in both the “condition” and “control” groups; a value greater than 1 indicates that a negative result is more likely in the “condition” group; and a value less than 1 indicates that a negative result is more likely in the “control” group. In the case of an odds ratio, a value of 1 indicates that a positive result is equally likely among subjects in both the condition” and “control” groups; a value greater than 1 indicates that a positive result is more likely in the “condition” group; and a value less than 1 indicates that a positive result is more likely in the “control” group.

In the case of an AUC ROC value, this is computed by numerical integration of the ROC curve. The range of this value can be 0.5 to 1.0. A value of 0.5 indicates that a classifier (e.g., a DNA methylation status) is no better than a 50% chance to classify unknowns correctly between two groups of interest, while 1.0 indicates the relatively best diagnostic accuracy. In certain embodiments, an epigenetic marker or panel of markers, including at least one epigenetic marker, is selected to exhibit a positive or negative likelihood ratio of at least about 1.5 or more or about 0.67 or less, at least about 2 or more or about 0.5 or less, at least about 5 or more or about 0.2 or less, at least about 10 or more or about 0.1 or less, or at least about 20 or more or about 0.05 or less.

In certain embodiments, an epigenetic marker or panel of markers, including at least one epigenetic marker, is selected to exhibit an odds ratio of at least about 2 or more or about 0.5 or less, at least about 3 or more or about 0.33 or less, at least about 4 or more or about 0.25 or less, at least about 5 or more or about 0.2 or less, or at least about 10 or more or about 0.1 or less.

In certain embodiments, an epigenetic marker or panel of markers, including at least one epigenetic marker, is selected to exhibit an AUC ROC value of greater than 0.5, preferably at least 0.6, more preferably 0.7, still more preferably at least 0.8, even more preferably at least 0.9, and most preferably at least 0.95.

In some cases, multiple thresholds may be determined in so-called “tertile”, “quartile”, or “quintile” analyses. In these methods, for example, the “diseased (e.g., carcinoma)” and “control groups” (or “high risk” and “low risk”) groups are considered together as a single population, and are divided into 3, 4, or 5 (or more) “bins” having equal numbers of individuals. The boundary between two of these “bins” may be considered “thresholds.” A risk (of a particular diagnosis or prognosis for example) can be assigned based on which “bin” a test subject falls into.

In other embodiments, particular thresholds for the DNA methylation status of an epigenetic marker or panel of epigenetic markers are not relied upon to determine if the DNA methylation status obtained from a subject are correlated to a particular diagnosis or prognosis. For example, a temporal change in the DNA methylation status of an epigenetic marker or panel of epigenetic markers can be used to rule in or out one or more particular diagnoses and/or prognoses. Alternatively, the DNA methylation statuses of an epigenetic marker or panel of epigenetic markers are correlated to a condition, disease, prognosis, treatment efficacy etc., by the presence or absence of a DNA methylation status of an epigenetic marker or panel of epigenetic markers in a particular assay format. In the case of epigenetic marker panels, the present invention may utilize an evaluation of the entire profile of epigenetic markers to provide a single result value (e.g., a “panel response” value expressed either as a numeric score or as a percentage risk).

In certain embodiments, a panel of epigenetic markers is selected to assist in distinguishing a pair of groups (i.e., assist in assessing whether a subject has an increased likelihood of being in one group or the other group of the pair) selected for example from “healthy condition” and “carcinoma”, “a first stage or severity of carcinoma” and “a second stage or severity of carcinoma”, or “low risk” and “high risk” with at least about 70%, 80%, 85%, 90% or 95% sensitivity, suitably in combination with at least about 70% 80%, 85%, 90% or 95% specificity. In some embodiments, both the sensitivity and specificity are at least about 75%, 80%, 85%, 90% or 95%.

The phrases “assessing the likelihood” and “determining the likelihood”, as used herein, refer to methods by which the skilled artisan can predict the presence or absence of a condition (e.g., a condition selected from healthy condition, carcinoma, a particular stage of carcinoma, or a particular severity of carcinoma) in a patient. The skilled artisan will understand that this phrase includes within its scope an increased probability that a condition is present or absence in a patient; that is, that a condition is more likely to be present or absent in a subject. For example, the probability that an individual identified as having a specified condition actually has the condition may be expressed as a “positive predictive value” or “PPV.” Positive predictive value can be calculated as the number of true positives divided by the sum of the true positives and false positives. PPV is determined by the characteristics of the predictive methods of the present invention as well as the prevalence of the condition in the population analyzed. The statistical algorithms can be selected such that the positive predictive value in a population having a condition prevalence is in the range of 70% to 99% and can be, for example, at least 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In other examples, the probability that an individual identified as not having a specified condition actually does not have that condition may be expressed as a “negative predictive value” or “NPV.” Negative predictive value can be calculated as the number of true negatives divided by the sum of the true negatives and false negatives. Negative predictive value is determined by the characteristics of the diagnostic or prognostic method, system, or code as well as the prevalence of the disease in the population analyzed. The statistical methods and models can be selected such that the negative predictive value in a population having a condition prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, a subject is determined as having a significant likelihood of having or not having a specified condition. By “significant likelihood” is meant that the subject has a reasonable probability (0.6, 0.7, 0.8, 0.9 or more) of having, or not having, a specified condition (e.g., healthy condition, carcinoma, a stage of carcinoma or severity of carcinoma).

The DNA methylation status analysis of the present invention permits the generation of data sets that can be evaluated using informatics approaches. Informatics analytical methods are known and software is available to those in the art, e.g., cluster analysis (Pirouette, Informetrix), class prediction (SIMCA-P, Umetrics), principal components analysis of a computationally modeled dataset (SIMCA-P, Umetrics), 2D cluster analysis (GeneLinker Platinum, Improved Outcomes Software), and metabolic pathway analysis (biotech.icmb.utexas.edu). The choice of software packages offers specific tools for questions of interest (Kennedy et al., Solving Data Mining Problems Through Pattern Recognition. Indianapolis: Prentice Hall PTR, 1997; Golub et al., (2999) Science 286:531-7; Eriksson et al., Multi and Megavariate Analysis Principles and Applications: Umetrics, Umea, 2001). In general, any suitable mathematic analysis can be used to evaluate the DNA methylation status of at least one (e.g., 1, 2, 3, 4, 5, 6, 7, 8, 9, 10, et.) epigenetic marker with respect to a condition selected from healthy condition, carcinoma, a particular stage of carcinoma, or a particular severity of carcinoma. For example, methods such as multivariate analysis of variance, multivariate regression, and/or multiple regression can be used to determine relationships between dependent variables (e.g., clinical measures) and independent variables (e.g., DNA methylation status). Clustering, including both hierarchical and non-hierarchical methods, as well as nonmetric Dimensional Scaling can be used to determine associations or relationships among variables and among changes in those variables.

In addition, principal component analysis is a common way of reducing the dimension of studies, and can be used to interpret the variance-covariance structure of a data set. Principal components may be used in such applications as multiple regression and cluster analysis. Factor analysis is used to describe the covariance by constructing “hidden” variables from the observed variables. Factor analysis may be considered an extension of principal component analysis, where principal component analysis is used as parameter estimation along with the maximum likelihood method. Furthermore, simple hypothesis such as equality of two vectors of means can be tested using Hotelling's T squared statistic.

In some embodiments, the data sets corresponding to a DNA methylation status of an epigenetic marker or to a DNA methylation status profile of more than one epigenetic marker are used to create a diagnostic or predictive rule or model based on the application of a statistical and machine learning algorithm. Such an algorithm uses relationships between the DNA methylation status of an epigenetic marker or panel of epigenetic markers and a condition selected from healthy condition, carcinoma, a particular stage of carcinoma, or a particular severity of carcinoma observed in control subjects or typically cohorts of control subjects (sometimes referred to as training data), which provides combined control or reference DNA methylation statuses for comparison with the DNA methylation status of an epigenetic marker or with a DNA methylation status profile of more than one epigenetic marker in a nucleic acid sample obtained from a subject. The data are used to infer relationships that are then used to predict the status of a subject, including the presence or absence of one of the conditions referred to above.

The term “correlating” generally refers to determining a relationship between one type of data with another or with a state. In various embodiments, correlating a DNA methylation status of an epigenetic marker or a DNA methylation status profile of more than one epigenetic marker with the presence or absence of a condition (e.g., a condition selected from a healthy condition, carcinoma, a particular stage of carcinoma, or a particular severity of carcinoma) comprises determining the presence, absence or level of DNA methylation in at least one epigenetic marker in a biological sample obtained from a subject that suffers from that condition; or in persons known to be free of that condition. In specific embodiments, a profile of DNA methylation levels, absences or presences is correlated to a global probability or a particular outcome, using receiver operating characteristic (ROC) curves.

### 4. Method of Treatment

The diagnostic methods of the present invention are also suitable for identifying patients that may require treatment; that is, patient stratification.

Thus, another aspect of the present invention provides a method of treating a carcinoma in a subject, the method comprising:

analyzing the DNA methylation status of the MED15 promoter in a biological sample obtained from the subject;

determining the presence of the carcinoma in the subject or an increased likelihood that a carcinoma is present in the subject based on the analysis; and

exposing the subject to a treatment regimen for treating the carcinoma.

The biological samples can be analyzed at the point of care or they can be sent to laboratories to conduct the analysis. Thus, in a related aspect, the present invention provides a method of treating a carcinoma in a subject, the method comprising:

(a) sending a biological sample obtained from the subject to a laboratory to have an assay conducted, wherein the assay comprises analyzing the DNA methylation status of the MED15 promoter in the biological sample; and determining the presence of the carcinoma in the subject or an increased likelihood that a carcinoma is present in the subject based on the analysis;

(b) receiving the results of the assay of step (a); and

(c) exposing the subject to a treatment regimen for treating the carcinoma if the results indicate that the subject has or has an increased likelihood of having a carcinoma.

The term “treating” as used herein, unless otherwise indicated, means reversing, alleviating, inhibiting the progress of, or preventing, either partially or completely, the growth of the carcinoma, tumor metastasis, or other cancer-causing or neoplastic cells in a patient. The term “treating” as used herein, unless otherwise indicated, also means alleviating, inhibiting the progress of, or preventing, either partially or completely, the symptoms associated with a carcinoma, such as organ failure, pain or any other symptoms known to persons skilled in the art as being associated with a carcinoma. The term “treatment” as used herein, unless otherwise indicated, refers to the act of treating.

As used herein, the term “treatment regimen” refers to prophylactic and/or therapeutic (i.e., after onset of a specified condition) treatments, unless the context specifically indicates otherwise. The term “treatment regimen” encompasses natural substances and pharmaceutical agents (i.e., “drugs”) as well as any other treatment regimen including but not limited to dietary treatments, physical therapy, exercise regimens, surgical interventions, radiation therapy and combinations thereof.

Following diagnosis, treatment is often decided according to the type of carcinoma, its anatomical location in the subject and its size (i.e., its stage). The “stage” of a carcinoma is a descriptor (usually numbers I to IV) of how much the carcinoma has spread. The stage often takes into account the size of a primary and/or secondary tumor, how deep it has penetrated, whether it has invaded adjacent organs, if and how many lymph nodes it has metastasized to, and whether it has spread to distant organs. Staging of a carcinoma is important because the stage at diagnosis is a predictor of survival, and treatments are often changed based on the stage.

Thus, the present invention contemplates exposing the subject to a treatment regimen if the subject tests positive for the presence or likelihood of the presence of the carcinoma. Non-limiting examples of such treatment regimens include radiotherapy, surgery, chemotherapy, hormone ablation therapy, pro-apoptosis therapy and immunotherapy.

Radiotherapies include radiation and waves that induce DNA damage for example, γ-irradiation, X-rays, UV irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half life of the isotope, the strength and type of radiation emitted, and the uptake by the neoplastic cells.

Non-limiting examples of radiotherapies include conformal external beam radiotherapy (50-100 Grey given as fractions over 4-8 weeks), either single shot or fractionated, high dose rate brachytherapy, permanent interstitial brachytherapy, systemic radio-isotopes (e.g., Strontium 89). In some embodiments the radiotherapy may be administered in combination with a radiosensitizing agent. Illustrative examples of radiosensitizing agents include but are not limited to efaproxiral, etanidazole, fluosol, misonidazole, nimorazole, temoporfin and tirapazamine.

Chemotherapeutic agents may be selected from any one or more of the following categories:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyridines like 5-fluorouracil and tegafur, raltitrexed, methotrexate, cytosine arabinoside and hydroxyurea; anti-tumor antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like paclitaxel and docetaxel; and topoisomerase inhibitors (for example epipodophyllotoxins like etoposide and teniposide, amsacrine, topotecan and camptothecin);

(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), UH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) agents which inhibit cancer cell invasion (for example metalloproteinase inhibitors like marimastat and inhibitors of urokinase plasminogen activator receptor function);

(iv) inhibitors of growth factor function, for example such inhibitors include growth factor antibodies, growth factor receptor antibodies (for example the anti-erbb2 antibody trastuzumab [Herceptin™] and the anti-erbbl antibody cetuximab [C225]), farnesyl transferase inhibitors, MEK inhibitors, tyrosine kinase inhibitors and serine/threonine kinase inhibitors, for example other inhibitors of the epidermal growth factor family (for example other EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazoli-n-4-amine (CI 1033)), for example inhibitors of the platelet-derived growth factor family and for example inhibitors of the hepatocyte growth factor family;

(v) anti-angiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);

(vi) vascular damaging agents such as Combretastatin A4 and compounds disclosed in International Patent Applications WO 99/02166, WO00/40529, WO 00/41669, WO01/92224, WO02/04434 and WO02/08213;

(vii) antisense therapies, for example those which are directed to the targets listed above, such as ISIS 2503, an anti-ras antisense; and

(viii) gene therapy approaches, including for example approaches to replace aberrant genes such as aberrant p53 or aberrant GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy.

Immunotherapy approaches, include for example ex-vivo and in-vivo approaches to increase the immunogenicity of patient tumor cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumor cell lines and approaches using anti-idiotypic antibodies. These approaches generally rely on the use of immune effector cells and molecules to target and destroy cancer cells. The immune effector may be, for example, an antibody specific for some marker on the surface of a malignant cell. The antibody alone may serve as an effector of therapy or it may recruit other cells to actually facilitate cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a malignant cell target. Various effector cells include cytotoxic T cells and NK cells.

Examples of other cancer therapies include phototherapy, cryotherapy, toxin therapy or pro-apoptosis therapy. One of skill in the art would know that this list is not exhaustive of the types of treatment modalities available for cancer and other hyperplastic lesions.

As hereinbefore described, the diagnostic potential of the method of the present invention may be improved by analyzing additional markers that are predictive of the presence of the carcinoma or an increased likelihood that a carcinoma is present in the subject. Thus, in some embodiments disclosed herein, the method of treatment further comprises analyzing the DNA methylation status of the promoter of one or more genes selected from the group consisting of DAPK1, p16^{INK4α} and RASSF1α in a biological sample obtained from the subject. In other embodiments disclosed herein, the method of treatment further comprises analyzing the DNA methylation status of the promoter of one or more genes selected from the group consisting of DAPK1, p16^{INK4α}, RASSF1α and TIMP3 in a biological sample obtained from the subject. In still other embodiments disclosed herein, the method of treatment further comprises analyzing the DNA methylation status of the promoter of one or more genes selected from the group consisting of p16^{INK4α}, RASSF1α and TIMP3 in a biological sample obtained from the subject.

In some embodiments, the presence of the carcinoma or an increased likelihood that a carcinoma is present in the subject is based on increased methylation of the MED15 promoter and increased methylation of the promoter of the one or more genes selected from the group consisting of DAPK1, p16^{INK4α} and RASSF1α when compared to the level of methylation of the same promoter in a non-cancerous cell from the same subject.

In other embodiments, the presence of the carcinoma or an increased likelihood that a carcinoma is present in the subject is based on increased methylation of the MED15 promoter and increased methylation of the promoter of the one or more genes selected from the group consisting of DAPK1, p16′, RASSF1α and TIMP3 when compared to the level of methylation of the same promoter in a non-cancerous cell from the same subject.

In still other embodiments, the presence of the carcinoma or an increased likelihood that a carcinoma is present in the subject is based on increased methylation of the MED15 promoter and increased methylation of the promoter of the one or more genes selected from the group consisting ofp16′, RASSF1α and TIMP3 when compared to the level of methylation of the same promoter in a non-cancerous cell from the same subject.

In some embodiments, the method of treatment further comprises analyzing the DNA methylation status at a CpG cluster of the MED15 promoter region. In yet another embodiment, the CpG cluster is located at position 20,861,680 to 20,862,252 of human chromosome 22. In yet another embodiment, the method of treatment further comprises analyzing the DNA methylation status at the 5′ end of the CpG cluster. In yet a further embodiment, the method further comprises analyzing the DNA methylation status at the 3′ end of the CpG cluster.

### 5. Method of Monitoring Treatment

The present invention can also be used to monitor the efficacy of treatment for a carcinoma or a symptom thereof. Thus, in another aspect of the present invention, there is provided a method for monitoring efficacy of a treatment regimen in a subject with a carcinoma, the method comprising:

analyzing the DNA methylation status of the MED15 promoter in a biological sample obtained from the subject; and

monitoring the subject over a period of time for a change in the methylation status of the MED15 promoter region,

wherein a change or otherwise in the methylation status of the MED15 promoter over the period of time is indicative of treatment efficacy.

In some embodiments, the methods comprise the analysis of a series of biological samples obtained over a period of time from approximately the same anatomical location (e.g., saliva or buccal cell scrape from the same area of the mouth cavity). In another embodiment, the method comprises analyzing a series of biological samples obtained over a period of time from different anatomical locations or by analyzing a series of biological samples obtained over a period of time from a combination of the same and different anatomical locations.

It would be understood by persons skilled in the art that a reduction in the level of methylation of the MED15 promoter over the period of time is indicative of effective treatment. Conversely, it would be understood that no change or an increase in the level of methylation of the MED15 promoter over the period of time is indicative of ineffective treatment.

As hereinbefore described, it would be understood by persons skilled in the art that the diagnostic specificity and sensitivity of the methods of the present invention may be improved by using a panel or combination of markers (i.e., in addition to the analysis of the DNA methylation status of the MED15 promoter region). For example, at least 1, 2, 3, 4, 5, 6, 7, 8, 9, 10 or more additional markers can be used in combination with the diagnostic method of the present invention. Thus, some embodiments disclosed herein, step (a) further comprise analyzing the DNA methylation status of the promoter of a gene selected from the group consisting of DAPK1, p16^{INK4α} and RASSF1α, and step (b) further comprises monitoring the patient over a period of time for a change in the methylation status of the promoter of the one or more genes selected from the group consisting of DAPK1, p16^{INK4α} and RASSF1α, wherein a change or otherwise in the methylation status of the MED15 promoter and a change or otherwise in the methylation status of the promoter of the one or more genes selected from the group consisting of DAPK1, p16^{INK4α} and RASSF1α over the period of time is indicative of treatment efficacy. In other embodiments disclosed herein, step (a) further comprise analyzing the DNA methylation status of the promoter of a gene selected from the group consisting of DAPK1, p16^{INK4α}, RASSF1α and TIMP3, and step (b) further comprises monitoring the patient over a period of time for a change in the methylation status of the promoter of the one or more genes selected from the group consisting of DAPK1, p16^{INK4α}, RASSF1α and TIMP3, wherein a change or otherwise in the methylation status of the MED15 promoter and a change or otherwise in the methylation status of the promoter of the one or more genes selected from the group consisting of DAPK1, p16^{INK4α}, RASSF1α and TIMP3over the period of time is indicative of treatment efficacy. In still other embodiments disclosed herein, step (a) further comprise analyzing the DNA methylation status of the promoter of a gene selected from the group consisting of p16^{INK4α}, RASSF1α and TIMP3, and step (b) further comprises monitoring the patient over a period of time for a change in the methylation status of the promoter of the one or more genes selected from the group consisting of p16^{INK4α}, RASSF1α and TIMP3, wherein a change or otherwise in the methylation status of the MED15 promoter and a change or otherwise in the methylation status of the promoter of the one or more genes selected from the group consisting of p16^{INK4α}, RASSF1α and TIMP3over the period of time is indicative of treatment efficacy.

It would be understood by persons skilled in the art that a reduction in the level of methylation of the promoter of the one or more genes selected from the group consisting of DAPK1, p16^{INK4α}, RASSF1α and TIMP3 over the period of time is indicative of effective treatment. Conversely, it would be understood that no change or an increase in the level of methylation of the promoter of the one or more genes selected from the group consisting of DAPK1, p16^{INK4α}, RASSF1α and TIMP3 in the MED15 promoter over the period of time is indicative of ineffective treatment.

In some embodiments disclosed herein, where there has been no change or an increase in the level of methylation at the MED15 promoter over the period of time, the method further comprises increasing the dose of treatment given to the subject. This may comprise administering to the subject additional doses of the same agent with which they are being treated or changing the dose and/or type of medication. Where the subject is being treated by radiotherapy, increasing the dose of treatment given to the subject may comprise applying higher dose of radiation and/or more frequent doses of radiation. It may also comprise combining the subjects current radiotherapy with a chemotherapeutic agent that can be administered by any suitable route (e.g. intravenously, orally).

In some embodiments, where there has been a reduction in the level of methylation at the MED15 promoter over the period of time, the method further comprises reducing the dose of treatment given to the subject. This may be particularly advantageous where current treatment has resulted in unwanted side effects, such that a reduction in the dose of treatment may reduce the unwanted side effects.

In some embodiments, the method of monitoring a subject being treated for a carcinoma further comprises increasing the dose of treatment given to the subject where no change or an increase in the level of methylation of the promoter of the one or more genes selected from the group consisting of DAPK1, p16^{INK4α} and RASSF1α over the period of time. In other embodiments, the method of monitoring a subject being treated for a carcinoma further comprises increasing the dose of treatment given to the subject where no change or an increase in the level of methylation of the promoter of the one or more genes selected from the group consisting of DAPK1,p16^{INK4α}, RASSF1α and TIMP3 over the period of time. In still other embodiments, the method of monitoring a subject being treated for a carcinoma further comprises increasing the dose of treatment given to the subject where no change or an increase in the level of methylation of the promoter of the one or more genes selected from the group consisting of p16^{INK4α}, RASSF1α and TIMP3 over the period of time.

In some embodiments, where there has been a reduction in the level of methylation of the promoter of the one or more genes selected from the group consisting of DAPK1, p16^{INK4α} and RASSF 1 a over the period of time, the method further comprises reducing the dose of treatment given to the subject.

The DNA methylation status of an epigenetic marker or panel of epigenetic markers disclosed herein further enables determination of endpoints in pharmacotranslational studies. For example, clinical trials can take many months or even years to establish the pharmacological parameters for a medicament to be used in treating a carcinoma or a particular stage or severity of a carcinoma (e.g., a squamous cell carcinoma, including head and neck squamous cell carcinoma). However, these parameters may be associated with a DNA methylation status of an epigenetic marker or panel of epigenetic markers associated with a health state (e.g., a healthy condition). Hence, the clinical trial can be expedited by selecting a treatment regimen (e.g., medicament and pharmaceutical parameters), which results in a DNA methylation status of an epigenetic marker or panel of epigenetic markers associated with the desired health state (e.g., healthy condition). This may be determined for example by (1) providing a correlation of a reference DNA methylation status or reference DNA methylation status profile with the likelihood of having the healthy condition; (2) obtaining a sample DNA methylation status or sample DNA methylation status profile from a subject having a carcinoma after treatment with a treatment regimen, wherein a similarity of the subject's DNA methylation status or DNA methylation status profile after treatment to the reference DNA methylation status or DNA methylation status profile indicates the likelihood that the treatment regimen is effective for changing the health status of the subject to the desired health state (e.g., healthy condition). This aspect of the present invention advantageously provides methods of monitoring the efficacy of a particular treatment regimen in a subject (for example, in the context of a clinical trial) already diagnosed with a carcinoma or with a particular stage or severity of carcinoma. These methods take advantage of DNA methylation status or DNA methylation status profiles that correlate with treatment efficacy, for example, to determine whether the DNA methylation status or DNA methylation status profile of a subject undergoing treatment partially or completely normalizes during the course of or following therapy or otherwise shows changes associated with responsiveness to the therapy.

Thus, the invention provides methods of correlating a reference DNA methylation status or DNA methylation status profile with an effective treatment regimen for a condition selected from a carcinoma or a particular stage or severity of a carcinoma (e.g., a squamous cell carcinoma, including head and neck squamous cell carcinoma), wherein the reference DNA methylation status profile evaluates the DNA methylation status of at least two (e.g., 2, 3, 4, 5, 6, 7, 8, 9, 10, etc.) epigenetic markers. These methods generally comprise: (a) determining a sample DNA methylation status or DNA methylation status profile from a subject with the condition prior to treatment (i.e., baseline); and correlating the sample DNA methylation status or DNA methylation status profile with a treatment regimen that is effective for treating that condition.

The invention further provides methods of determining whether a treatment regimen is effective for treating a subject with a condition selected from a carcinoma or a particular stage or severity of a carcinoma (e.g., a squamous cell carcinoma, including head and neck squamous cell carcinoma). These methods generally comprise: (a) correlating a reference DNA methylation status or DNA methylation status profile prior to treatment (i.e., baseline) with an effective treatment regimen for the condition, wherein the reference DNA methylation status profile evaluates the DNA methylation status of at least two (e.g., 2, 3, 4, 5, 6, 7, 8, 9, 10, etc.) epigenetic markers; and (b) obtaining a sample DNA methylation status or DNA methylation status profile from the subject after treatment, wherein the sample DNA methylation status or DNA methylation status profile after treatment indicates whether the treatment regimen is effective for treating the condition in the subject.

The invention can also be practiced to evaluate whether a subject is responding (i.e., a positive response) or not responding (i.e., a negative response) to a treatment regimen. This aspect of the invention provides methods of correlating a DNA methylation status or DNA methylation status profile with a positive and/or negative response to a treatment regimen. These methods generally comprise: (a) obtaining a sample DNA methylation status or DNA methylation status profile from a subject with a condition selected from a carcinoma or a particular stage or severity of a carcinoma (e.g., a squamous cell carcinoma, including head and neck squamous cell carcinoma) following commencement of the treatment regimen, wherein the reference DNA methylation status profile evaluates the DNA methylation status of at least two (e.g., 2, 3, 4, 5, 6, 7, 8, 9, 10, etc.) epigenetic markers; and (b) correlating the sample DNA methylation status or DNA methylation status profile from the subject with a positive and/or negative response to the treatment regimen.

The invention also provides methods of determining a positive and/or negative response to a treatment regimen by a subject with a condition selected from a carcinoma or a particular stage or severity of a carcinoma (e.g., a squamous cell carcinoma, including head and neck squamous cell carcinoma). These methods generally comprise: (a) correlating a reference DNA methylation status or DNA methylation status profile with a positive and/or negative response to the treatment regimen, wherein the reference DNA methylation status profile evaluates the DNA methylation status of at least two (e.g., 2, 3, 4, 5, 6, 7, 8, 9, 10, etc.) epigenetic markers; and (b) determining a sample DNA methylation status or DNA methylation status profile from the subject, wherein the subject's sample DNA methylation status or DNA methylation status profile indicates whether the subject is responding to the treatment regimen.

In some embodiments, the methods further comprise determining a first sample DNA methylation status or DNA methylation status profile from the subject prior to commencing the treatment regimen (i.e., a baseline profile), wherein the first sample DNA methylation status profile evaluates at least two (e.g., 1, 2, 3, 4, 5, 6, 7, 8, 9, 10, etc.) epigenetic markers; and comparing the first sample DNA methylation status or DNA methylation status profile with a second sample DNA methylation status or DNA methylation status profile from the subject after commencement of the treatment regimen.

This aspect of the invention can be practiced to identify responders or non-responders relatively early in the treatment process, i.e., before clinical manifestations of efficacy. In this way, the treatment regimen can optionally be discontinued, a different treatment protocol can be implemented and/or supplemental therapy can be administered. Thus, in some embodiments, a sample DNA methylation status or DNA methylation status profile is obtained within about 2 hours, 4 hours, 6 hours, 12 hours, 1 day, 2 days, 3 days, 4 days, 5 days, 1 week, 2 weeks, 3 weeks, 4 weeks, 6 weeks, 8 weeks, 10 weeks, 12 weeks, 4 months, six months or longer of commencing the treatment regimen.

### 6. Kits

In another aspect of the present invention, there is provided a kit for detecting the presence of a carcinoma or an increased likelihood that a carcinoma is present in a subject, or for monitoring efficacy of a treatment regimen in a subject with a carcinoma, or for evaluating whether a subject is responding or not responding to a treatment regimen for treating a carcinoma, or for determining a positive and/or negative response to a treatment regimen by a subject with a carcinoma, suitably using the methods described herein, the kit comprising at least one agent for detecting or quantifying the DNA methylation status of the MED15 promoter region.

The invention provides compositions and kits for analyzing the DNA methylation status of epigenetic markers as described herein. These kits may contain reagents for performing DNA methylation specific assays. Kits for carrying out the methods of the present invention typically include, in suitable container means, (i) a reagent for methylation specific reaction or separation, (ii) a probe that comprises an antibody or nucleic acid sequence that specifically binds to the marker polypeptides or polynucleotides of the invention, (iii) a label for detecting the presence of the probe and (iv) instructions for how to measure the level of methylation. The container means of the kits will generally include at least one vial, test tube, flask, bottle, syringe and/or other container into which a first antibody specific for one of the polypeptides or a first nucleic acid specific for one of the polynucleotides of the present invention may be placed and/or suitably aliquoted. Where a second and/or third and/or additional component is provided, the kit will also generally contain a second, third and/or other additional container into which this component may be placed. Alternatively, a container may contain a mixture of more than one reagent, each reagent specifically binding a different marker in accordance with the present invention. The kits of the present invention will also typically include means for containing the reagents (e.g., nucleic acids, polypeptides etc.) in close confinement for commercial sale. Such containers may include injection and/or blow-molded plastic containers into which the desired vials are retained.

The kits may further comprise positive and negative controls, as well as instructions for the use of kit components contained therein, in accordance with the methods of the present invention.

In some embodiments, the kit comprises a set of nucleic acid primers capable of selectively amplifying methylated of the MED15 promoter region. Non-limiting examples of suitable primers are those listed in Table 1.

In order that the invention may be readily understood and put into practical effect, particular preferred embodiments will now be described by way of the following non-limiting examples.

### EXAMPLES

### Example 1

### Materials and Methods

### Study Design:

This study was approved by the University of Queensland Medical Ethical Institutional Board and by the Princess Alexandra Hospital Ethics Review Board. In the first instance, we collected paraffin embedded tissue sections from HNSCC (n=6) patients. Certified pathologist assisted in the identification of the tumor and normal sections on the tissue slides. All participants gave informed consent before sample collection. Healthy control subjects (n=25) without any clinical signs of cancer as well as HNSCC patients (n=24) at various clinical stages of cancer (stages II-IV), were included. The HNSCC patients were HPV negative and predominantly of Caucasian background with poorly to moderately differentiated SCC.

### Saliva Sample Collection and Processing:

DNA methylation of the MED15/PCQAP promoter was assayed in DNA isolated from whole mouth saliva (drool) from healthy controls (non-smokers) and HNSCC patients (smokers and non-smokers, including those who recently quit smoking). The subjects were asked to sit in a comfortable upright position and rinse their mouth with water to remove any food debris. The subjects were asked to tilt their heads down and maintain that position for about 2-5 minutes so as to allow saliva to pool in the mouth. Saliva samples were collected in Falcon tubes (50 mL, Greiner, Germany) and were transported on dry ice to the laboratory. Samples were then thawed at room temperature and centrifuged at 500 g at 4-° C. for 10 minutes. The supernatant was discarded and the cellular pellet was frozen at −80-° C. to be later used for methylation-specific polymerase chain reaction (MSP) analysis.

### DNA Extraction and Bisulfite Conversion of Saliva Samples:

DNA extraction and subsequent bisulfite conversion were carried out using the EpiTectPlus kit® (Qiagen GmbH) according to the manufacturer's instructions with the exception of a longer elution incubation time (10 minutes instead of 1 min) and the use of a larger elution volume (17 μL instead of 15 Bisulfite-converted DNA was eluted from the column in elution buffer (10 mM Tris-HCl, pH 8.0) and immediately used for the MSP or stored at −80° C. All the converted DNA samples were assessed for their DNA purity and quantified on a Nanodrop 1000 Spectrophotometer (Thermo Scientific, USA).

Extraction and Bisulfite Conversion of the DNA from FFPE Samples:

Formalin-fixed, paraffin-embedded (FFPE) tissue samples from HNSCC (n=6) patients in stages II to IV were retrieved from the Department of Anatomical Pathology at the Princess Alexandra Hospital in Woolloongabba. A pathologist identified and confirmed the normal tissue from the carcinoma on hematoxylin and eosin (H&E) stained slides. The FFPE tissue samples were serially cut into 5 sections at approximately 5 μm thick. Of the 5 sections, one was stained with H&E to be used as a reference slide.

Internal areas of the carcinoma and areas of normal tissue that were most distant to the carcinoma (to minimize cross-contamination; Controls) were removed and DNA extracted (coupled with bisulfite conversion) using an EpiTect Fast FFPE Bisulfite Kit (Qiagen, GmbH) according to the manufacturer's instructions. All bisulfite converted DNA samples were assessed for DNA purity and quantified on a Nanodrop 1000 Spectrophotometer (Thermo Scientific, USA). From ing to 25 ng of bisulfite converted DNA was used for downstream applications.

### Identification of the Methylation Sites in the MED15/PCQAP Promoter

In order to identify novel methylation sites, amplification of the main CpG clusters in the MED15/PCQAP promoter was performed from the bisulfite converted DNA samples of histologically-identified tumor and normal tissue using AmpliTaq Gold^{(r)} 360 Master Mix, CAT. 4398901proofreading polymerase mix (Applied Biosystems, USA). Primer design for bisulfite PCR of the CpG island and subsequent MSP screening were performed using the MethDB (urogene dot org/methprimer/) and BiSearch (bisearch dot enzim dot hu/) online computational resources.

Primers listed in Table 1 were used to amplify a region of approximately 700 bp between positions 20,861,600 and 20,862,400 of human chromosome 22 (GRCh37/hg19; see also FIG. 1. Panel A). Using primers listed in Table 1, first round of amplification was performed using the following cycling conditions: 95-° C. for 10 min, followed by 40 cycles of 95° C. for 30 s, 60° C. for 2 min, 72° C. for 1 min, followed by a final extension step for 7 min at 72° C. using a Bio-Rad thermal cycler. Direct sequencing of the products from 6 patient biopsy-derived samples was preceded by a second round of amplification adding sequencing-optimized adapter sequences (Table 1) was also performed with identical PCR conditions as first round of amplification followed by sequencing reaction using (Tag) primers. The sequencing reactions were carried in a 20 μL reaction 20% BigDye1.1 mix (ABI Biosystems); 17.5% sequencing buffer, 5% glycerol and amplified PCR product (after second round, approximately 10 ng) using the following conditions: 98° C. for 5 min, 30 cycles of 98° C. for 10 s, 50° C. for 30 s and 60° C. for 4 min.

Due to ambiguity in the methylation status at the CpG island preceding the 5′ CpG target cluster, an ambiguous base pair (Y) was introduced in the forward 5′ MSP primers (see Table 1).

### MSP Analysis of the MED15/PCQAP Methylation Status in HNSCC Samples

Specificity of the designed MSP primer pairs was confirmed on the unconverted DNA which resulted in no gene specific amplifications. Quantification of the MSP amplicons was performed using intensity measurements with an ChemiDoc gel imager and Image1.1 software (Bio-Rad, USA). The methylated, unmethylated and gDNA loading control PCRs were then quantified after running them on an agarose gel that was subsequently stained with GelRed DNA-binding dye. An “adjusted volume” value was used to quantify the MSPstatistical amplicons. The MSP was carried out as a one-stage amplification of 35 cycles (95° C. for 30 s, 62.5° C. for 30 s, 72° C. for 30 s), preceded by an incubation at 95° C. for 5 min, and followed by a final extension step for 10 min at 72° C., using a Bio-Rad thermal cycler.

### Statistical Analysis of the Results:

To assess the statistical significance of any difference in the methylation status of the MED15/PCQAP promoter in HNSCC patients and normal controls, an unpaired t-test with Welch's correction and non-parametric Mann-Whitney tests were utilized. Difference was considered significant at a stringent cut-off of p<0.01. Data point plots and receiver-operating characteristic (ROC) curves were generated using GraphpadPrism6 software and online tools (GraphPad, Inc. and graphpad dot com/quickcalcs) as well as logistic regression analysis using the R software package.

### Example 2

### Identification of HNSCC-Specific Methylation in MED15/PCQAP Promoter

Specific methylation patterns associated with HNSCC tumors were identified within the CpG island of the MED15/PCQAP promoter by amplifying bisulfite converted DNA from HNSCC patients and comparing this to amplified bisulfite converted DNA from normal tissue of a number of patients. Primers were designed to flank all of the CpG sites in the island (see Methods and Methods and Table 1) and were used to generate PCR products from formalin-fixed, paraffin-embedded (FFPE) tissue samples of HNSCC tumors, which were then used for determining methylation patterns. Two adjacent CpG clusters each demonstrated consistent tumor-specific methylation patterns, the first being doubly-methylated in 5 out of 6 HSCC patient samples, and the second in 4 out of 6 HNSCC patient samples (see FIG. 1. Panel B).

Sequence analysis also revealed several single nucleotide polymorphisms (SNPs) in this in all of 6 sequenced individual genomes (see FIG. 1).

### Example 3

### MSP Analysis of Methylation at the Novel Sites Within MED15/PCQAP Promoter

Upon identification of the differentially-methylated CpGs, MSP strategies were designed to reliably screen for the presence of both alleles. To achieve this, 3 primers were designed for each of the two CpG doublets (the 5′ and 3′ CpG clusters). The first common primer was designed within 200 bps from the target CpG, to work in a methylation-insensitive manner, while the other 2 primers, one for methylated and the other for unmethylated versions using MSP algorithms. Specificity of the primers was verified using an ePCR tool for bisulfite-converted DNA PCR prediction on human genome at the BiSearch portal (bisearch dot enzim dot hu/). The efficiency and specificity of the MSP were validated using near fully artificially CpG Methylated HeLa gDNA (New England BioLabs, UK) as a positive control. Tests were also conducted in bisulfite-converted gDNA from a pluripotent stem cell line and a blood leukocytic fraction as negative controls. An MSP amplicon (showing a strong signal by agarose gel electrophoresis) was obtained from HeLa DNA with methylated allele-specific MSP primer sets, while the unmethylated primer pairs were effective at amplifying negative controls.

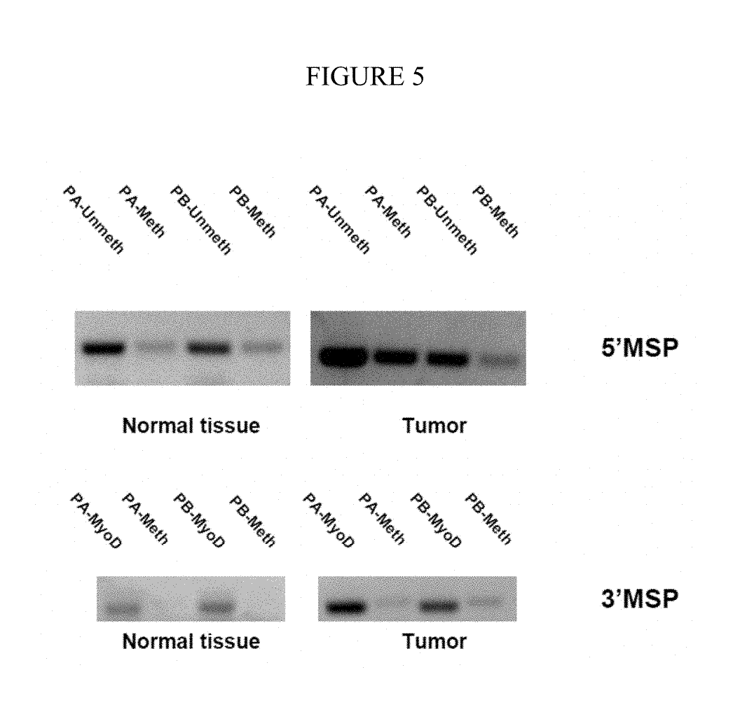

The results demonstrated significantly higher methylation of the MED15/PCQAP promoter in DNA samples obtained from FFPE carcinoma as compared to the level of methylation of the MED15/PCQAP promoter in DNA samples obtained from the adjacent normal FFPE tissues (see FIG. 5).

### Example 4

Methylation Levels at Novel Sites of the MED15/PCQAP Promoter are Significantly Elevated in DNA from the Saliva of HNSCC Patients

Quantitative analyses showed that the level of methylation of the MED15/PCQAP promoter in DNA from the saliva of HNSCC patients was significantly higher than the level of methylation of the MED15/PCQAP promoter in DNA from the saliva of healthy controls. For each of the saliva samples, quantification of relative methylation levels were performed by comparing the methylated and unmethylated forms of MSP amplicons of the two identified CpG clusters in the MED15/PCQAP promoter region (the 5′ and 3′ CpG clusters, as shown in FIG. 1).

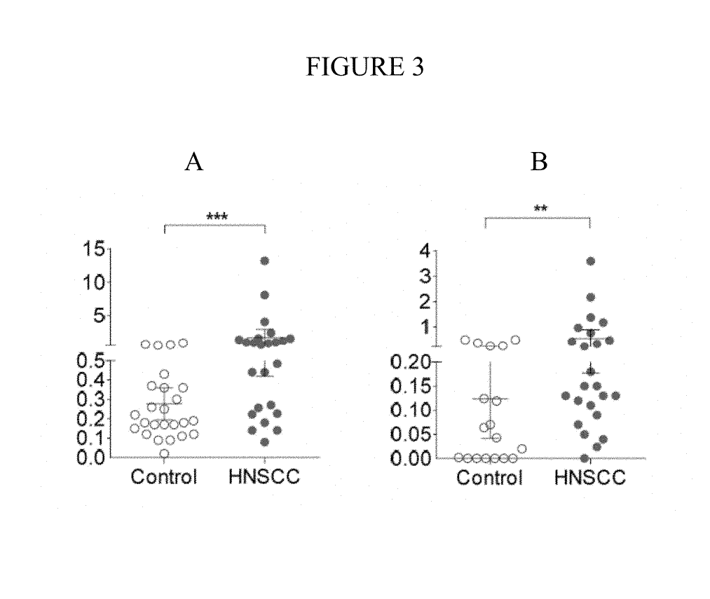

For the 5′ CpG cluster, the ratio of methylated to unmethylated forms for most HNSCC patients was at least 0.4 and for most controls, the ratio was less than 0.4 (see FIG. 3. Panel A). For the 3′ CpG cluster, the ratio of methylated to unmethylated forms for most HNSCC patients was at least 0.13 and for most controls, the ratio was less than 0.13 (see FIG. 3. Panel B). Analysis of the data using the non-parametric Mann-Whitney test yielded a P value of 0.0006 (Prism6 software, GraphPad, Inc.). The Kolmogorov-Smirnov test indicating very high significance with a P value of less than 0.01.

### Example 5

### Predictive Power of the New Simple MSP-Based Saliva Test

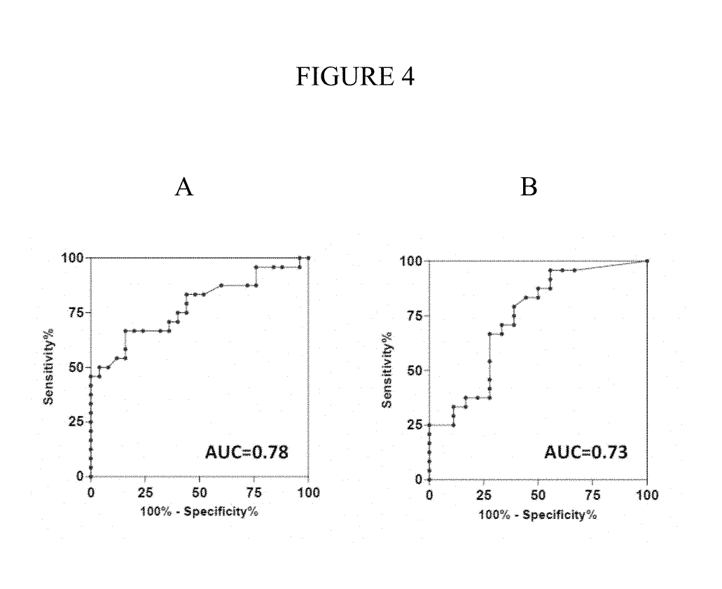

Receiver-operating characteristic (ROC) analysis was used to assess the methylation status of the MED15/PCQAP promoter as a tool for the diagnosis of HNSCC. To quantitate the performance of the MSP assay directed specifically to each of the two novel CpG clusters, standard sensitivity versus specificity plots was generated (see FIG. 4).

The ROC curve parameters were 0.78 and 0.73 for the 5′ and 3′ CpG clusters, respectively, indicating the ability of the methylation status of the MED15/PCQAP promoter to accurately identify the presence of HNSCC in a patient by MSP analysis on DNA from patient saliva.

### Example 6

### Predictive Power of Biomarker Panel Including MED15 Biomarkers

### Method

Three cohorts of study participants were recruited: (i) healthy control non-smokers (n=49); (2) healthy control smokers (at enrolment, subjects are 25 years or older with a cigarette smoking history of >20 pack years, n=20) and (3) HNSCC patients (both HPV-negative and HPV-positive patients, n=62 each). HNSCC patients were recruited from the Princess Alexandra Hospital (the largest head and neck cancer center in Queensland, Australia). Smoking participants were classified according to the WHO criteria as former smokers, never smokers or current smokers [22]. Clinical stages of the HNSCC patients were classified according to the TNM system of the American Joint Committee on Cancer. The sample sizes were based upon estimates from a pilot study conducted earlier. In that study, sample sizes were calculated in sets, designed specifically to detect differences between controls and HNSCC patients. The pilot study data were log transformed and the means and standard deviations were calculated. Sample sizes were calculated for each control x gene difference using a two-sample t test with pooled variance, two tails, an alpha or p value of 0.05, and a power of 0.80. Equal group size was assumed. The sample size sets were further adjusted for an estimated error rate (source unspecified) of 10%. Not included was an estimate of the difference between the control and HNSCC patients as the means were too close together and the sample size too large.

### Saliva Sample Collection and Processing:

DNA sample preparation, extraction and bisulfite conversion were carried out according to methods described above in Example 1.

### MSP Technology:

Nested MSP was carried out for RASSF1α and p16^{INK4α} using primers and methods described by Ovchinnikov et al. (7) and for TIMP3 using primers and methods described by Righini et al. (23). For RASSF1α and p16^{INK4α} amplification cycling conditions were: initial denaturing stage at 94° C. for 2 min, followed by 5 cycles of 15 s at 94° C., 15 s at 62° C. and 15 s at 72° C. with three repeats of decreasing annealing temperature (64° C., 62° C. and 60° C. in that order) before a final elongation stage at 72° C. for 5 min. In each of the stages of the PCR reactions, 1μL of PCR product was used as DNA template. For TIMP3 MSP amplification, a total PCR reaction volume of 10 μL was employed, including 5 μL of EmeraldAmp® MAX HS PCR Master Mix (Takara, Japan), 10 μM of respective primer sets (non-methylated and methylated), 20 ng of DNA template for methylated and 1 ng of DNA for non-methylated. The MSP PCR conditions were: initial denaturing stage at 95° C. for 5 min, followed by 40 cycles of 15 s at 94° C., 15 s at 54° C. and 15 s at 72° C., followed by 4 min at 72° C. as the final elongation step, using a Bio-Rad T100™ thermal cycler.

For both the MED15 5′ CpG and 3′ CpG sites, PCRs were carried out in a 12.5 volume with 2×EmeraldAmp® MAX HS PCR Master Mix (6.25 μL, Takara, Japan); Forward and Reverse end primer concentrations of 0.8 μM; 5% DMSO; 0.1 μg/mL of BSA and converted DNA template (1 ng for non-methylation/MyoD and 25 ng for methylation). 5′ CpG site MSP was carried out as a onestage amplification of 35 cycles (95° C. for 30 s, 62.5° C. for 30 s, 72° C. for 60 s), preceded by an incubation at 95° C. for 3 min, followed by a final extension step for 5 min at 72° C., using a Bio-Rad thermal cycler. In contrast, 3′ CpG site MSP was carried out as a onestage amplification of 35 cycles (95° C. for 20 s, 62.5° C. for 20 s, 72° C. for 30 s), preceded by an incubation at 95° C. for 3 min, and followed by a final extension step for 10 min at 72° C., using a Bio-Rad thermal cycler. Quantification of the MSP product levels was performed using intensity measurements with FUSION-SL chemiluminescence gel imager and Image J 1.47 software (Fiji software)). Methylated, unmethylated and gDNA loading control PCRs were quantified, after electrophoresis on a 2% agarose gel and staining with GelRed DNA-binding dye. “Integrated density” values were used to quantify PCR amplicons. Ratios of methylated to unmethylated forms of the 5′ CpG cluster as well as ratios of methylated to MyoD for 3′CpG were calculated.

### Results

The diagnosticpotential of the 5-marker panel was assessed using logistic regression analysis and the statistical software package R. The entire process was cross-validated. Sensitivity and specificity were calculated for a uniform prior. This may be interpreted as a form of shrinkage regularization, where the estimates are shrunken to lie in a reduced space. The sensitivity and specificity of each marker in the panel are presented in Table 2.

TABLE 2

| Methylation | Sensitivity | Specificity | ROC-AUC | |
|---|---|---|---|---|
| Marker | (%) | (%) | 95% CI | P Value |
| TIMP-3 | 75 | 70 | 0.76 (0.63-0.90) | P < 0.001 |
| PCQAP-5′ | 70 | 65 | 0.70 (0.58-0.80) | P < 0.01 |
| PCQAP-3′ | 65 | 63 | 0.76 (0.51-0.74) | P < 0.05 |
| p16 | 81 | 65 | 0.67 (0.64-0.89) | P < 0.0001 |
| RASSF1α | 73 | 66 | 0.74 (0.63-0.84) | P < 0.001 |

Cross-validated discriminant function scores were used to estimate a ROC curve. The ROC curve was calculated by moving a critical threshold along the axis of the discriminant function scores. Both raw empirical ROCs and smoothed ROCs were calculated using standard methods. Curves were calculated for comparison of healthy control smokers and HNSCC patients. The area under the curve (AUC) was calculated by the trapezoidal rule, applied to both the empirical ROC and the smoothed ROC.

The ROC curve provides a useful summary of the diagnostic potential of an assay. A perfect diagnostic assay has an ROC curve which is a horizontal line passing through the point with sensitivity and specificity both equal to one. The area under the ROC curve for such a perfect diagnostic is 1. A useless diagnostic assay has a ROC curve which is given by a 45 degree line through the origin. The area for such an uninformative diagnostic is 0.5.

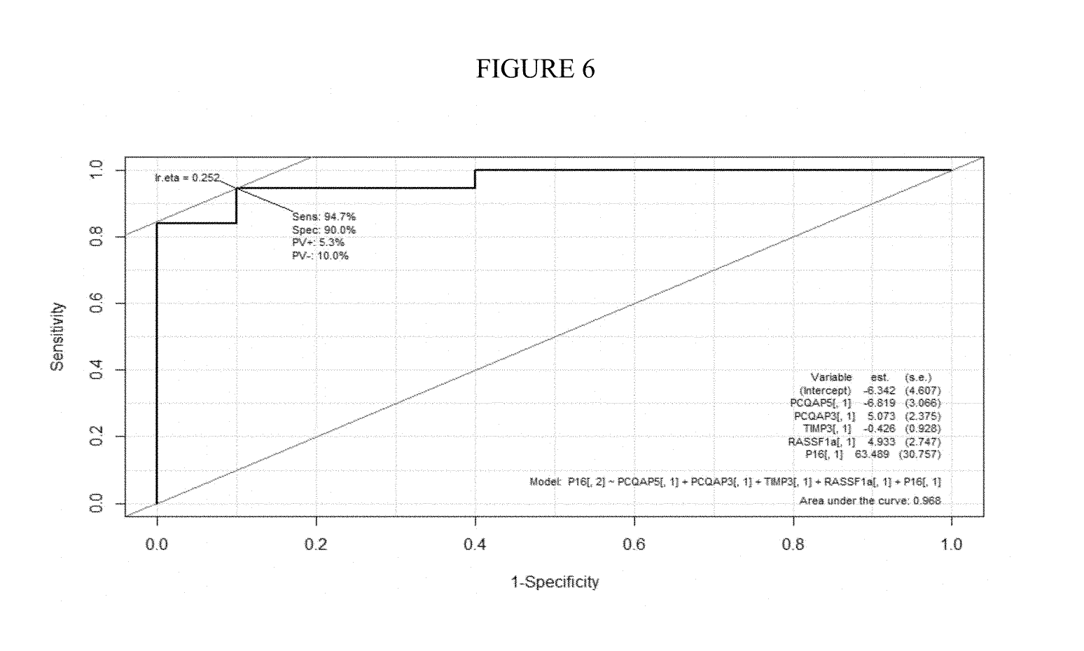

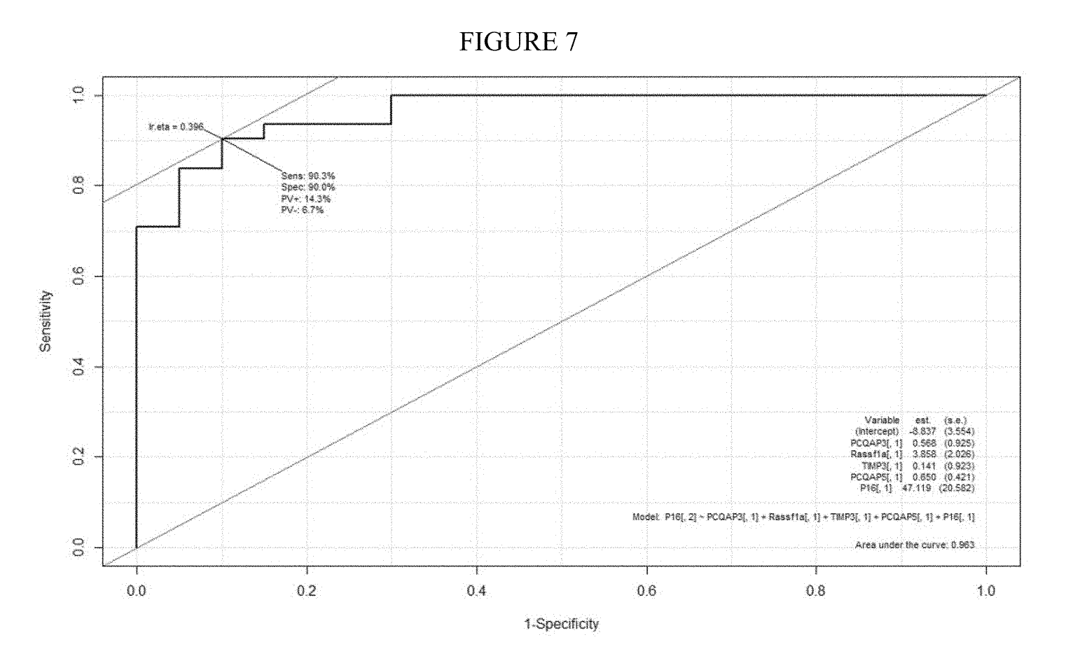

The ROC curve for the 5-marker in saliva MSP analysis based on a comparison between healthy control smokers and HNSCC patients is presented in FIG. 6, which shows the marker panel having an AUC of 0.97, a sensitivity of 95% and a specificity of 90%. The ROC curve for the 5-marker MSP in saliva analysis based on a comparison between healthy control subjects and HNSCC patients is presented in FIG. 7. The diagnostic capability of this panel as shown in this figure is very high: AUC=0.96 with a sensitivity and specificity of 90% each.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

1. Parkin D M, et al. 2005, CA Cancer J Clin; 55:74-108.
2. Boyle PaL, B. 2008, World Cancer Report; 510.
3. Sankaranarayanan Ret al. 1998, Anticancer Res; 18:4779-86.
4. Thomas S J, et al. 2007, Int J Cancer; 120:1318-23.
5. Zhao M, et al. 2012, J Mol Cell Biol; 5:57-60.
6. Laird P W. 2010, Nature Reviews Genetics; 11:191-203.
7. Ovchinnikov et al. 2012, Translational Oncology; 5(5):321-326.
8. Toyota et al., 1999, Cancer Res.; 59:2307-2312.
9. Melnikov et al, 2005, Nucleic Acids Res; 33(10):e93.
10. Frommer et al., 1992, Proc. Natl. Acad. Sci. USA; 89:1827-1831.
11. Sambrook, J., et al., Molecular Cloning: A Laboratory Manual, (2001) 3rd edition, Cold Spring Harbor, N.Y.
12. Gonzalgo and Jones, 1997, Nucleic Acids Res.; 25:2529-2531.
13. Eads et al., 2000, Nucleic Acid Res. 28:e 32.
14. Cottrell et al., 2007, J. Urology 177, 1753.
15. Cottrell et al., 2004, Nuc. Acids Res. 32, e10.
16. Wojdacz and Dobrovic, 2007, Nuc. Acids Res. 35(6):e41.
17. Wojdacz et al., 2008, Nat. Prot. 3(12):1903-1908.
18. Balic et al., 2009, J. Mol. Diagn. 11:102-108.
19. Candiloro et al., 2011, Epigenetics 6(4):500-507.
20. Sun et al., 2012, Clin. Cancer Res. 18(4):1082-1091.
21. Sun et al., 2012, PLoS One 7(3):e33642.
22. Prevention., U.C.f.D.C.a., Health behaviors of adults: United States 2005-2007. Vital and Health Statistics, 2010. Number 245: p. 80
23. Righini et al., 2007, Clin. Cancer Res. 13(4):1179-1185.

1.-46. (canceled)

47. A composition comprising a DNA sample from a patient having or suspected of having head and neck squamous cell carcinoma (HNSCC), a DNA polymerase and primers for amplification of a CpG site in the MED15 gene promotor.

48. A composition according to claim 47, wherein the DNA sample comprises uracil bases in the DNA.

49. A composition according to claim 47, further comprising a molecule that fluoresces in the presence of double stranded DNA.

50. A composition according to claim 49, wherein the molecule is SYBR-green.

51. A composition according to claim 47, wherein the primers are specific for a methylated version of the CpG site.

52. A composition according to claim 47, wherein the primers are specific for an unmethylated version of the CpG site.

53. A composition according to claim 47, further comprising a fluorescent probe specific for a methylated version of the CpG site.

54. A composition according to claim 47, further comprising a fluorescent probe specific for an unmethylated version of the CpG site.

55. A composition according to claim 47, further comprising probes and primers specific for one or more CpG sites in the promoters of one or more gene selected from the group comprising RASSF1α, TIMP3, DAPK1 and P16^{INK4α}.

56. A composition according to claim 47, wherein the primers are specific for amplification of one or more CpG sites located within a region of DNA at position 20,861,680 to 20,862,252 of human Chromosome 22.

57. A composition according to claim 47, wherein the DNA sample comprises uracil bases in the DNA and wherein the primers are specific for amplification of one or more CpG sites located within a region of DNA at position 20,861,680 to 20,862,252 of human Chromosome 22.

58. A composition according to claim 57, further comprising a probe specific for a methylated version of the CpG site.

59. A composition according to claim 57, further comprising a probe specific for an unmethylated version of the CpG site.

60. A composition according to claim 57, wherein the head and neck squamous cell carcinoma (HNSCC) is an oral squamous cell carcinoma (OSCC).

61. A composition according to claim 47, wherein the head and neck squamous cell carcinoma (HNSCC) is an oral squamous cell carcinoma (OSCC).
